SEQUENCE LISTING (2 sequences)

SEQ ID NO: 1 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 2 - protein, 569 aa, Homo sapiens
MKVLLRLICFIALLISSLEADKCKEREEKIILVSSANEIDVRPCPLNPNEHKGTITWYKDDSKTPVSTEQASRIHQHKEKLWFVPAKVEDSGHYYCVVRNSSYCLRIKISAKFVENEPNLCYNAQAIFKQKLPVAGDGGLVCPYMEFFKNENNELPKLQWYKDCKPLLLDNIHFSGVKDRLIVMNVAEKHRGNYTCHASYTYLGKQYPITRVIEFITLEENKPTRPVIVSPANETMEVDLGSQIQLICNVTGQLSDIAYWKWNGSVIDEDDPVLGEDYYSVENPANKRRSTLITVLNISEIESRFYKHPFTCFAKNTHGIDAAYIQLIYPVTNFQKHMIGICVTLTVIIVCSVFIYKIFKIDIVLWYRDSCYDFLPIKASDGKTYDAYILYPKTVGEGSTSDCDIFVFKVLPEVLEKQCGYKLFIYGRDDYVGEDIVEVINENVKKSRRLIIILVRETSGFSWLGGSSEEQIAMYNALVQDGIKVVLLELEKIQDYEKMPESIKFIKQKHGAIRWSGDFTQGPQSAKTRFWKNVRYHMPVQRRSPSSKHQLLSPATKEKLQREAHVPLG

(19) United States
(12) Patent Application Publication
(10) Pub. No.: US 2023/0398150 A1
(43) Pub. Date: Dec. 14, 2023

(54) SPECIFIC TARGETING OF TUMOR-INFILTRATING REGULATORY T CELLS (TREGS) USING ICOS AND IL-1R

(71) Applicant: FRED HUTCHINSON CANCER CENTER, Seattle, WA (US)

(72) Inventors: Martin Prlic, Seattle, WA (US); Florian Mair, Seattle, WA (US); Jami R. Erickson, Seattle, WA (US)

(73) Assignee: FRED HUTCHINSON CANCER CENTER, Seattle, WA (US)

(21) Appl. No.: 18/249,149

(22) PCT Filed: Oct. 14, 2021
(86) PCT No.: PCT/US2021/054978

§ 371 Date: Apr. 14, 2023

Related U.S. Application Data
(60) Provisional application No. 63/092,957, filed on Oct. 16, 2020.

(51) Int. Cl. A61K 35/17; C07K 16/28; A61P 35/00; A61K 39/00; A61K 45/06

(52) U.S. Cl. CPC A61K 35/17; C07K 16/2818; C07K 16/2866; A61P 35/00; A61K 39/4631; A61K 39/464419; A61K 39/464429; A61K 39/4611; A61K 45/06

(57) ABSTRACT

The disclosure provides compositions and related methods for detecting, inhibiting, reducing or killing tumor-infiltrating regulatory T cells (Tregs) characterized by expression of inducible T cell costimulator (ICOS) and Interleukin-1 receptor type 1 (IL-1R1). The reagents can include a bi-specific affinity reagent with a first domain that specifically binds ICOS and a second domain that specifically binds IL-1R1 or, separately, a first affinity reagent that specifically binds ICOS and a second affinity reagent that specifically binds IL-1R1. In some embodiments, the reagent can comprise engineered immune cells expressing a first chimeric antigen receptor (CAR) specific for ICOS and a second CAR specific for IL-1R1, wherein the cell requires binding by the first CAR and second CAR to activate, such as a logic-gated CAR T cell.

### CROSS-REFERENCE TO RELATED APPLICATION

This application claims the benefit of U.S. Provisional Application No. 63/092,957, filed Oct. 16, 2020, the disclosure of which is incorporated herein by reference in its entirety.

### STATEMENT REGARDING SEQUENCE LISTING

The sequence listing associated with this application is provided in text format in lieu of a paper copy and is hereby incorporated by reference into the specification. The name of the text file containing the sequence listing is 1896-P37WO_Seq_List_FINAL_20211012_ST25.txt. The text file is 11 KB; was created on Oct. 12, 2021; and is being submitted via EFS-Web with the filing of the specification.

### BACKGROUND

Cells of both the adaptive and innate immune system can take up permanent residence in non-lymphoid tissues, which profoundly changes their phenotype and function relative to their circulating counterparts. A well-studied example is tissue-resident memory T cells (T_{RM}), which perform important immunosurveillance function in many human peripheral tissues and have been shown to adapt site-specific transcriptional and functional signatures. Both T_{RM} cells and other subsets of tissue-resident immune cells are poised to rapidly produce pro-inflammatory cytokines after stimulation, and exposure to inflammatory cues can drastically change the composition and functional properties of the local immune subsets.

Cells with a T_{RM} phenotype, together with a wide range of other adaptative and innate immune cells are also present in a large proportion of human solid tumor types. The composition of this immune infiltrate is a critical determinant of tumor development as well as disease progression. In particular tumor-infiltrating regulatory T cells (Tregs) are considered a major factor for the inefficient immune responses seen in many solid tumors. Tregs are thought to be a main driver of the immunosuppressive environment that prevents the rejection of solid tumors by the immune system. Depletion of Tregs from the tumor microenvironment is an attractive therapeutic target, but there are currently no biomarkers that allow selective targeting of Tregs in solid tumors. Importantly, systemic system depletion of Tregs is not feasible as it leads to severe autoimmunity.

Functionally exhausted T cells are considered a major factor for the inefficient immune responses seen in many solid tumors. Various studies have focused on the identification of drivers for T cell exhaustion, suggesting that the transcription factors TCF1 and TOX are critical for the loss of effector function in exhausted CD8⁺ cytotoxic T cells infiltrating solid tumors. Importantly, the checkpoint molecule programmed-death 1 (PD-1), which is highly expressed by exhausted CD8⁺ T cells has been the target of various therapeutic approaches. However, PD-1 is also expressed by a large fraction of T_{RM} cells, and can be induced by exposure to inflammatory common-gamma-chain cytokines such as IL-2 or IL-15.

Another key immune component in the tumor microenvironment are myeloid antigen-presenting cells (APCs), including dendritic cells (DCs) as well as macrophages and other monocyte-derived cells. These innate APCs are present at much lower numbers than adaptive immune cells, but are critical in initiating and sustaining T cell responses via expression of antigen-loaded MHC class I and II molecules, co-stimulatory and co-inhibitory molecules as well as secretion of a variety of cytokines and chemokines. Seminal studies utilizing high-dimensional single-cell analysis techniques provided critical insight into tumor-infiltrating APCs, revealing the presence of all canonical myeloid subpopulations in human lung cancer, and that prior classifications into M1/M2 phenotypes might be too simplistic. Furthermore, very recent work identified a novel DC subset in non-small cell lung cancer (NSCLC), characterized by elevated expression of immunoregulatory molecules which was dubbed mregDCs.

Despite all these substantial advances to illuminate immune cell function in the tumor microenvironment, two critical roadblocks hamper a better understanding as to why efficient anti-tumor immune responses are not developing in many cases, and why only a fraction of patients responds to typical immunotherapeutic intervention. First, studies of the immune infiltrate in human tumors have so far relied on comparison to either peripheral blood or healthy steady-state tissue biopsies, and contrary to the murine model system there is scarce data in humans how an inflammatory response changes the local immune milieu in non-lymphoid tissues. Thus, it remains unclear which of the immune phenotypes seen in tumor tissues are the result of generalized inflammation in the tumor microenvironment as opposed to tumor-specific adaptation. Untangling these two intertwined processes could not only reveal novel targets for more efficacious anti-tumor therapies, but also help designing methods that avoid the sometimes serious side-effects of systemic checkpoint blockade.

Second, few studies have performed parallel profiling of APCs and T cells. While it is well established that APC-T cell interactions initiate, sustain and shape the subsequent T cell response, the nature of these interactions remain poorly defined in human tissues, both during non-malignant inflammatory processes as well as tumor tissues. Defining tumor-unique ligand-receptor signaling events could not only help us better understand the development of T cell exhaustion, but open up novel therapeutic avenues by targeting signaling networks that are only found in tumor tissues, and not during general inflammatory processes.

Accordingly, despite the advances in characterizing immune cell function in solid tumors, there remains a need to identify tumor specific phenotypes of immune cells to facilitate their manipulation in effective anti-tumor treatments. The present disclosure addresses these and related needs.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the disclosure provides a method of specifically inhibiting or depleting solid tumor-infiltrating regulatory T cells (Tregs). The method comprises contacting the solid tumor with one or more agents that specifically bind inducible T cell costimulator (ICOS) and Interleukin-1 receptor type 1 (IL-1R1).

In some embodiments, the one or more agents comprises a bi specific affinity reagent with a first domain that specifically binds ICOS and a second domain that specifically binds IL-1R1. In some embodiments, the one or more agents comprises a first affinity reagent that specifically binds ICOS and a second affinity reagent that specifically binds IL-1R1. In some embodiments, the one or more agents induces Treg cell death. In some embodiments, the one or more agents is conjugated to a payload that is toxic to the tumor-infiltrating Tregs. In some embodiments, the one or more agents comprise an engineered immune cell that co-expresses a first chimeric antigen receptor (CAR) that specifically binds ICOS and a second chimeric antigen receptor (CAR) that specifically binds IL-1R1, wherein the engineered immune cell requires binding by the first CAR and second CAR to activate. In some embodiments, the engineered immune cell is a logic-gated CAR T cell that requires binding of the first CAR and the second CAR to induce a T cell response by the CAR T cell. In some embodiments, the one or more agents comprise a logic-gated CAR T cell that co-expresses a first chimeric antigen receptor (CAR) that specifically binds one of ICOS and IL-1R1, and a second chimeric antigen receptor (CAR) that specifically binds to the other of ICOS and IL-1R1 via a bi-functional switch molecule, wherein the CAR T cell requires binding by the first CAR and second CAR to induce a T cell response by the CAR T cell. In some embodiments, the method further comprises contacting the solid tumor with an effective amount of the bi-functional switch molecule, wherein the bi-functional switch molecule comprises a first domain that specifically binds to the other of ICOS and IL-1R1 and a second domain that is specifically bound by the second CAR. In some embodiments, the inhibiting or depleting the Tregs in the solid tumor reduces immunosuppressive conditions in the solid tumor.

In another aspect, the disclosure provides a method of treating a subject with a solid tumor. The method comprises administering to the subject a therapeutic composition comprising one or more agents that specifically bind inducible T cell costimulator (ICOS) and Interleukin-1 receptor type 1 (IL-1R1).

In some embodiments, the one or more agents comprises a bi-specific affinity reagent with a first domain that specifically binds ICOS and a second domain that specifically binds IL-1R1. In some embodiments, the one or more agents comprises a first affinity reagent that specifically binds ICOS and a second affinity reagent that specifically binds IL-1R1. In some embodiments, the one or more agents bind to solid tumor-infiltrating regulatory T cells Tregs and cause cell death of the Tregs in the solid tumor. In some embodiments, the one or more agents is conjugated to a payload that is toxic to the tumor-infiltrating Tregs. In some embodiments, the one or more agents comprise an engineered immune cell that co-expresses a first chimeric antigen receptor (CAR) that specifically binds ICOS and a second chimeric antigen receptor (CAR) that specifically binds IL-1R1, wherein the engineered immune cell requires binding by the first CAR and second CAR to activate. In some embodiments, the engineered immune cell is a logic-gated CAR T cell that requires binding of the first CAR and the second CAR to induce a T cell response by the CAR T cell. In some embodiments, the one or more agents comprise a logic-gated CAR T cell that co-expresses a first chimeric antigen receptor (CAR) that specifically binds one of ICOS and IL-1R1, and a second chimeric antigen receptor (CAR) that specifically binds to the other of ICOS and IL-1R1 via a bi-functional switch molecule, wherein the CAR T cell requires binding by the first CAR and second CAR to induce a T cell response by the CAR T cell. In some embodiments, the method further comprises contacting the solid tumor with an effective amount of the bi-functional switch molecule, wherein the bi-functional switch molecule comprises a first domain that specifically binds to the other of ICOS and IL-1R1 and a second domain that is specifically bound by the second CAR. In some embodiments, the method further comprises administering to the subject an additional cancer therapy. In some embodiments, the additional cancer therapy comprises administration of a checkpoint inhibitor compound, an adoptive cell therapy, an anti-cancer antigen antibody or therapeutic composition. In some embodiments, the checkpoint inhibitor inhibits PD-1, PD-L1, CTLA-4, LAG-3, Tim-3, or TIGIT. In some embodiments, the immune checkpoint inhibits PD-1 and is selected from Pembrolizumab (Keytruda), Nivolumab (Opdivo), Cemiplimab (Libtayo), and the like; the immune checkpoint inhibits PD-L1 and is selected from Atezolizumab (Tecentriq), Avelumab (Bavencio), Durvalumab (Imfinzi), and the like; or the immune checkpoint inhibits CTLA-4 and is selected from Ipilimumab (Yervoy), and the like.

In some embodiments, the adoptive cell therapy comprises immune cells that improve immune response against the tumor. In some embodiments, the immune cells comprise T cells or NK cells that are genetically modified to express a chimeric antigen receptor (CAR) that specifically binds a tumor associate antigen. In some embodiments, the anti-cancer antigen antibody or therapeutic composition is selected from aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, duocarmycin, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (Taxol™), pilocarpine, prochloroperazine, rituximab, saproin, tamoxifen, taxol, topotecan hydrochloride, trastuzumab, vinblastine, vincristine, vinorelbine tartrate, and the like.

In some embodiments, the solid tumor is a squamous cell carcinoma (SCC) or a breast cancer tumor.

In another aspect, the disclosure provides a composition comprising an engineered immune cell that co-expresses a first chimeric antigen receptor (CAR) that specifically binds ICOS and a second chimeric antigen receptor (CAR) that specifically binds IL-1R1. The engineered immune cell requires binding by the first receptor and second receptor to activate.

In some embodiments, the engineered immune cell is a logic-gated CAR T cell that requires binding of the first CAR and the second CAR to induce a T cell response by the CAR T cell. In some embodiments, the composition is formulated for systemic administration.

In another aspect, the disclosure provides a composition comprising a logic-gated CAR T cell that co-expresses a first chimeric antigen receptor (CAR) that specifically binds one of ICOS and IL-1R1, and a second chimeric antigen receptor (CAR) that specifically binds to the other of ICOS and IL-1R1 via a bi-functional switch molecule, wherein the CAR T cell requires binding by the first CAR and second CAR to induce a T cell response by the CAR T cell. In some embodiments, the bi-functional switch molecule comprises a first domain that specifically binds to the other of ICOS and IL-1R1 and a second domain that is specifically bound by the second CAR, and the CAR T cell requires simultaneous binding by the first domain to the other of ICOS and IL-1R1 and the second domain to the second CAR to induce a T cell response by the CAR T cell.

In another aspect, the disclosure provides a method of detecting the presence of tumor-infiltrating Treg cells in a tumor environment, comprising:
contacting a sample comprising tumor cells obtained from a subject with a solid tumor with one or more agents that specifically bind inducible T cell costimulator (ICOS) and Interleukin-1 receptor type 1 (IL-1R1), wherein the one or more agents are detectably labeled; and
detecting binding of the one or more agents to a cell in the sample, wherein binding the one or more agents to a cell in the sample indicates the presence of tumor-infiltrating Treg cells in the tumor environment in the subject.

In some embodiments, the one or more agents are agents comprises a bi specific affinity reagent with a first domain that specifically binds ICOS and a second domain that specifically binds IL-1R1. In some embodiments, the one or more agents comprises a first affinity reagent that specifically binds ICOS and a second affinity reagent that specifically binds IL-1R1. In some embodiments, the first affinity reagent produces a first detectable signal and the second affinity reagent produces a second affinity signal that is different from the first detectable signal. In some embodiments, the detecting binding of the one or more agents to a cell in the sample comprises flow cytometry. In some embodiments, the method further comprises treating the subject with a determined presence of tumor-infiltrating Treg cells in the tumor environment with a treatment to inhibit or deplete the tumor-infiltrating Treg cells.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIGS. 1A-1E: The CD4⁺ helper and CD8⁺ cytotoxic T cell phenotypes in SCC show large phenotypic overlap with inflamed reference tissues. (1A) Overview of the experimental strategy. Inflamed oral mucosal tissue samples were collected during routine dental surgeries, and oral squamous cell carcinoma (SCC) samples were from treatment-naive patients after surgical resection of the tumor. For each patient, matched peripheral blood samples were collected. (1B) Quantification of CD3⁺ T cells, CD19⁺ B cells and CD56⁺ NK cells (left panels) as well as the frequency of CD4⁺ and CD8⁺ T cells (right panels) across the indicated tissue sources. (1C) Representative plots showing the expression pattern for the tissue residency markers CD69 and CD103 on CD8⁺ T cells across peripheral blood, oral mucosa, and oral tumor (SSC) samples. Quantification of CD69+ CD103+ as well as CD69⁺ CD103⁺ cells is shown on the right. (1D) Representative plots and quantification of the expression for PD-1 (left), the transcription factor TCF-1 (middle) and the effector molecule Granzyme B (right) across the indicated tissue sources. (1E) Heatmap representing the expression pattern for all the indicated molecules within CD8⁺ cytotoxic T cells (left panel) as well as CD4⁺ helper T cells (without Tregs, right panel) across peripheral blood, oral mucosa, and oral tumor samples. Color coding indicates the percentage of positive cells for the respective marker.

FIGS. 2A-2E: Computational analysis using FAUST reveals a tumor-specific Treg phenotype co-expressing HLA-DR and ICOS. (2A) The top T cell phenotypes showing differential abundance between oral mucosal tissues and oral tumor tissues as identified by FAUST. Negative markers are not listed. (2B) Representative plots showing the frequency of CD25⁺ CD127⁻ Tregs across peripheral blood, oral mucosa, and oral tumor (SSC) samples. Quantification of the Treg population within CD4⁺ T cells and total T cells is shown on the right. (2C) Representative plots showing the expression pattern for the Treg markers Foxp3, CTLA4, CD39 and TIGIT across CD4⁺ CD25⁺ CD127⁻ Tregs and CD4⁺ CD25⁻ helper T cells in the tumor as well as peripheral blood. (2D) Representative plots showing the expression pattern of ICOS and HLA-DR across the three different tissue sources for CD4⁺ CD25⁺ CD127⁻ Tregs (upper panel) and CD4⁺ CD25⁻ helper T cells (lower panel). Quantification of the ICOS⁺ HLADR⁺ population as well as the ICOS⁺ HLADR⁻ population is shown on the right. (2E) Expression pattern and quantification of PD1 and CD69 for the DN, ICOS⁻ HLADR⁺, ICOS⁺ HLADR⁻, and ICOS⁺ HLADR⁺ Treg population in tumor tissues.

FIGS. 3A-3F: The APC compartment in the SCC microenvironment shows large phenotypic heterogeneity and an activated cDC2 phenotype (3A) Representative general gating strategy for the identification of canonical myeloid antigen-presenting cells (APCs) in oral squamous cell carcinoma (SCC) tissues. (3B) Quantification of the indicated cell populations relative to total CD45⁺ live cells (for CD14⁺ cells and Lin⁻ HLADR⁺ cells) and relative to the Lin⁻ HLADR⁺ fraction (for CD123⁺ pDCs, CD141⁺ cDC1 s, cCD1c⁺ cDC2s, and CD16⁺ and CD68⁺ cells). (3C) Representative histograms showing the expression pattern of the indicated phenotyping markers across (from top to bottom) CD14⁺ monocytes/macrophages, CD123⁺ pDCs, CD141⁺ cDC1s, CD1c⁺ cDC2s, and ON cells. (3D) Heatmap representing the expression pattern for all the indicated molecules within CD1c⁺ cDC2s (top panel), CD141⁺ cDC1s (middle panel) as well as CD14⁺ cells (lower panel) across peripheral blood, oral mucosa, and oral tumor samples. (3E) Expression pattern for CD206, CD163 and CX3CR1 across the indicated subsets and tissue origins. (3F) Representative plots and quantification of CD80⁺ cells within cDC2s (left panel) as well as CD40⁺ PD-L1⁺ cDC2s.

FIGS. 4A-4H: Comprehensive single-cell RNAseq analysis of SCC and inflamed reference tissues reveals subset-specific cytokine modules in the APC compartment. (4A) UMAP plots of the combined single-cell RNAseq data after Harmony integration colored by donor (left plot) and colored by cell annotation as defined by SingleR (right panel). (4B) UMAP plot colored by tissue origin of the cells (blood, mucosa, and tumor). (4C) UMAP plot of the myloid APC populations, colored by cluster (left panel) and showing key differentially expressed genes per cluster on a z-score normalized heatmap (right). (4D) Relative cluster abundance across the different donors and tissue sources. (4E) Relative contribution of each tissue source to the indicated cell cluster. (4F) Dot Plot showing the expression pattern of the indicated transcripts across the different myeloid cell populations. Size of the dot represents how many of the cells in a cluster express a given transcript, and color scheme indicates the average expression level. (4G) Number of differentially expressed (“DE”) genes between tumor and mucosa-derived cells in a given cellular cluster. (4H) Violin Plots showing the expression of key molecules for the DC3 cluster (left panel) and the cDC1 cluster (right panel).

FIGS. 5A-5E: NicheNet analysis reveals subset-specific crosstalk between tumor-infiltrating myeloid APCs and T cells and a distinct L-1/IL-1 R1 signaling axis to regulatory T cells (Tregs). (5A) Simplified overview of the NicheNet workflow. (5B) Circos plots showing the top20 ligand-receptor pairs between myeloid APCs and CD4⁺ helper T cells (left), CDS⁺ cytotoxic T cells (middle) and CD4⁺ Tregs (right). Transparency of the connection represents the interaction strength, and the ligands are colored as cytokine/co-receptors, other molecules, and ligands that were unique to a given T cell subset. (5C) Representative plots showing the expression for the cytokines IL-lb and IL-1a of the indicated APC subsets after ex vivo culture in the presence of Brefeldin A. (5D) Representative plots as well as quantification for the expression of the IL-1 receptor type 1 (IL-1 R1) in the indicated T cell subsets in blood and tumor. (5E) Representative plots showing that within total CD45⁺ live immune cells in the tumor, the majority (80-90%) of the ICOS⁺ IL-1R1⁺ cell fraction falls within the CD4⁺ CD25⁺ CD127⁻ Treg gate.

FIGS. 6A-6E: IL-1R1-expressing Tregs represent a functionally distinct Treg population in the human tumor microenvironment. (6A) UMAP plot of T cells sorted from three different SCC donors after performing a targeted transcriptomics experiment, colored by cluster with manual annotation. (6B) Heatmap showing key differentially expressed genes per cluster on a z-score normalized heatmap (right). (6C) Violin plots showing the expression profile of general Treg marker genes as well as IL-1R1⁺ Treg unique genes on blood-derived Tregs, tumor-derived IL-1R1⁻ Tregs and IL-1R1⁺ Tregs. (6D) Expression of the chemokine receptors CCR8 and CXCR6 on the indicated T cell populations (left plots). Histogram overlays show expression of ICOS and IL-1R1 on the Treg subsets based on CXCXR6 and CCR8 expression. (6E) Stratification of TCGA survival data for HNSCC (left) as well as breast cancer (right) by high (red) and low (red) expression of IL-1R1.

FIGS. 7A and 7B: Analysis of immune infiltrate in solid breast tumor tissue for expression of ICOS and IL-1R1. (7A) Tumor-infiltrating leukocytes were isolated from a human breast cancer tissue as described for SCC tumor samples. Plots depict gating for CD4⁺ and CD8⁺ T cells, and CD25⁺ CD127⁻ regulatory T cells (Tregs), followed by the expression pattern of ICOS and IL-1R1 on Tregs. (7B) Histogram plots show absence of IL-1R1 expression on tumor-infiltrating CD8⁺ T cells and CD4⁺ non Tregs, and expression of IL-1R1 on approximately 30% of Tregs (cut-off indicated by dashed line).

FIGS. 8A-8C: Intratumoral IL-1R1-expressing Tregs represent a clonally expanded Treg population with hallmarks of recent TCR activation and superior suppressive capacity. (8A) IL-1R1 expression on sorted Tregs from peripheral blood of healthy donors (“no IL-1R1”) and HNSCC tumor tissue cultured unstimulated or in the presence of anti-CD3/28 beads for 2 days. TCR stimulation was sufficient to induce IL-1R1 expression. (8B) Analysis of TCR diversity by single-cell VDJ sequencing within sorted IL-1R1⁺ Tregs from HNSCC tumors relative to total Tregs from matched peripheral blood. Every TCR sequence that was present in 2 cells or more was considered an expanded clone. (8C) Cell Trace Violet (CTV) dilution of sorted CD8⁺ T effector cells (Teff) derived from HNSCC tumor tissue after 4 days of culture without stimulation, with stimulation beads alone, or with an equal number of sorted tumor-derived IL-1R1⁺ and IL-1R1⁺ Tregs. Right plot depicts the percentage of divided T effector cells in each condition. All summary graphs are represented as mean±SD (total n=3 for stimulation assay and VDJ-sequencing, n=5 from multiple experiments for suppression assay). Statistical analyses were performed using one-way ANOVA with Tukey' s multiple comparisons test.

### DETAILED DESCRIPTION

Tissue residence and prolonged exposure to inflammation profoundly affect lymphocyte and myeloid cell function. Immune cells in solid tumors are thought to undergo additional adaptation, and the identification of tumor-driven unique immune cell alterations would allow for more efficient and precise tumor therapies.

As described in more detail below, this disclosure is based on the inventors' multi-omic approach to analyze the immune landscape of human oral squamous cell carcinomas (SCC) and inflamed non-malignant oral tissues in an effort to identify tumor-unique immune alterations and immune cell interactions. The inventors found substantial phenotypic congruence of the immune infiltrate in both tissue types, including exhausted T cells as well as recently described mregDCs. By combining computational and machine learning analysis methods, tumor-unique subsets of regulatory T cells (Tregs) and DC3s were identified. Analysis of the signaling networks between these cells provides an explanation for Treg accumulation in the tumor and identifies inducible T cell costimulatory (ICOS, also known as CD278) and Interleukin 1 receptor type 1 (IL-1R1) as unique cell surface protein markers that are not co-expressed by other immune populations in the blood or tumor. Moreover, this unique expression profile of tumor-infiltrating Treg cells provides an opportunity for therapeutic strategy (e.g. using so-called logic-gated CAR-T cells requiring both antigen targets for activation) to delete specifically tumor-infiltrating Tregs, but sparing circulating Tregs as well as other effector T cells.

In accordance with the foregoing, in one aspect, the present disclosure provides a method of specifically inhibiting or depleting solid tumor-infiltrating regulatory T cells (Tregs). The method comprises contacting the solid tumor with one or more agents that specifically bind inducible T cell costimulator (ICOS) and Interleukin-1 receptor type 1 (IL-1R1).

The one or more agents are typically affinity reagents that specifically bind to ICOS and IL-1R1. An exemplary, non-limiting ICOS protein has an amino acid sequence as set forth in SEQ ID NO:1, or a sequence with at least about 75%, 80%, 85%, 90%, 95%, or 98% identity thereto. An exemplary, non-limiting IL-1R1 protein has an amino acid sequence as set forth in SEQ ID NO:2, or a sequence with at least about 75%, 80%, 85%, 90%, 95%, or 98% identity thereto.

In some embodiments, the one or more agents specifically bind to an extracellular domain of ICOS and IL-1R1. An exemplary extracellular domain of ICOS has an amino acid sequence of residues 21-40 of SEQ ID NO:1, or a sequence with at least about 75%, 80%, 85%, 90%, 95%, or 98% identity thereto. An exemplary extracellular domain of IL-1R1 has an amino acid sequence of residues 18-336 of SEQ ID NO:2, or a sequence with at least about 75%, 80%, 85%, 90%, 95%, or 98% identity thereto. It will be understood that the affinity reagents can bind to specific epitopes within the extracellular domain and not the entire domain. As used herein, the term “specifically bind” or variations thereof refer to the ability of the affinity reagent(s) to bind to the antigen of interest (e.g., ICOS and IL-1R1), without significant binding to other molecules, under standard conditions known in the art.

Exemplary, non-limiting categories of affinity reagent include antibodies, an antibody-like molecule (including antigen-binding fragments of antibody fragments, derivatives), peptides that specifically interact with a particular antigen (e.g., peptibodies), antigen-binding scaffolds (e.g., DARPins, HEAT repeat proteins, ARM repeat proteins, tetratricopeptide repeat proteins, and other scaffolds based on naturally occurring repeat proteins, etc., [see, e.g., Boersma and Pluckthun, Curr. Opin. Biotechnol. 22:849-857, 2011, and references cited therein, each incorporated herein by reference in its entirety]), aptamers, or a functional ICOS and IL-1R1-binding domain or fragment thereof.

In some embodiments, the indicated affinity reagent is an antibody. As used herein, the term “antibody” encompasses antibodies and antigen binding antibody fragments or derivatives thereof, derived from any antibody-producing mammal (e.g., mouse, rat, rabbit, and primate including human), that specifically bind to an antigen of interest (e.g., ICOS and IL-1R1). Exemplary antibodies include multi-specific antibodies (e.g., bispecific antibodies); humanized antibodies; murine antibodies; chimeric, mouse-human, mouse-primate, primate-human monoclonal antibodies; and anti-idiotype antibodies. The antigen-binding molecule can be any intact antibody molecule or fragment or derivative thereof (e.g., with a functional antigen-binding domain).

An antibody fragment is a portion derived from or related to a full-length antibody, preferably including the complementarity-determining regions (CDRs), antigen binding regions, or variable regions thereof. Illustrative examples of antibody fragments and derivatives useful in the present disclosure include Fab, Fab′, F(ab)₂, F(ab′)₂ and Fv fragments, nanobodies (e.g., V_{H}H fragments and V_{NAR} fragments), linear antibodies, single-chain antibody molecules, multi-specific antibodies formed from antibody fragments, and the like. Single-chain antibodies include single-chain variable fragments (scFv) and single-chain Fab fragments (scFab). A “single-chain Fv” or “scFv” antibody fragment, for example, comprises the V_{H} and V_{L} domains of an antibody, wherein these domains are present in a single polypeptide chain. The Fv polypeptide can further comprise a polypeptide linker between the V_{H} and V_{L} domains, which enables the scFv to form the desired structure for antigen binding. Single-chain antibodies can also include diabodies, triabodies, and the like. Antibody fragments can be produced recombinantly, or through enzymatic digestion.

The above affinity reagents do not have to be naturally occurring or naturally derived, but can be further modified to, e.g., reduce the size of the domain or modify affinity for the ICOS and/or IL-1R1 as necessary. For example, complementarity determining regions (CDRs) can be derived from one source organism and combined with other components of another, such as human, to produce a chimeric molecule that avoids stimulating immune responses in a subject.

Production of antibodies or antibody-like molecules can be accomplished using any technique commonly known in the art. Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual (Cold Spring Harbor Laboratory Press, 2nd ed. 1988); Hammerling et al., in: Monoclonal Antibodies and T-Cell Hybridomas 563-681 (Elsevier, N.Y., 1981), incorporated herein by reference in their entireties. The term “monoclonal antibody” refers to an antibody that is derived from a single clone, including any eukaryotic, prokaryotic, or phage clone, and not the method by which it is produced. Methods for producing and screening for specific antibodies using hybridoma technology are routine and well known in the art. Bi-specific antibodies can incorporate CDR regions of two different identified monoclonal antibodies by fusing encoding gene portions for the relevant binding domains followed by cloning into an expression vector that also comprises nucleic acids encoding the remaining structure(s) of the bi-specific molecule.

Antibody fragments that recognize specific epitopes can be generated by any technique known to those of skill in the art. For example, Fab and F(ab′)₂ fragments of the invention can be produced by proteolytic cleavage of immunoglobulin molecules, using enzymes such as papain (to produce Fab fragments) or pepsin (to produce F(ab′)₂ fragments). F(ab′)₂ fragments contain the variable region, the light chain constant region and the CHI domain of the heavy chain. Further, the antibodies of the present invention can also be generated using various phage display methods known in the art.

The affinity reagent employed as the agent can also be an aptamer. As used herein, the term “aptamer” refers to oligonucleic or peptide molecules that can bind to specific antigens of interest. Nucleic acid aptamers usually are short strands of oligonucleotides that exhibit specific binding properties. They are typically produced through several rounds of in vitro selection or systematic evolution by exponential enrichment protocols to select for the best binding properties, including avidity and selectivity. One type of useful nucleic acid aptamers are thioaptamers, in which some or all of the non-bridging oxygen atoms of phophodiester bonds have been replaced with sulfur atoms, which increases binding energies with proteins and slows degradation caused by nuclease enzymes. In some embodiments, nucleic acid aptamers contain modified bases that possess altered side-chains that can facilitate the aptamer/ICOS or IL-1R1 binding.

Peptide aptamers are protein molecules that often contain a peptide loop attached at both ends to a protamersein scaffold. The loop typically has between 10 and 20 amino acids long, and the scaffold is typically any protein that is soluble and compact. One example of the protein scaffold is Thioredoxin-A, wherein the loop structure can be inserted within the reducing active site. Peptide aptamers can be generated/selected from various types of libraries, such as phage display, mRNA display, ribosome display, bacterial display and yeast display libraries.

In some embodiments, the one or more agents comprises a bi-specific affinity reagent with a first domain that specifically binds ICOS and a second domain that specifically binds IL-1R1. For example, the bi-specific affinity reagent can be a bi-specific antibody, or fragment or derivative thereof with a first domain that specifically binds ICOS and a second domain that specifically binds IL-1R1. In other embodiments, the one or more agents comprise at least two distinct affinity reagent molecules. For example, the one or more agents can comprise a first affinity reagent that specifically binds ICOS and a second affinity reagent that specifically binds IL-1R1. The first and second affinity reagent can be of the same general molecule type or different type (e.g., both can be antibody or antibody like molecules, or one can be an antibody and the other an aptamer, respectively).

In some embodiments, the one or more agents induces Treg cell death upon binding to ICOS and IL-1R1. For example, whether a single bi-specific affinity reagent that binds to both ICOS and IL-1R1 or two distinct mono-specific reagents that bind to ICOS and IL-1R1, respectively, the affinity reagent(s) can be conjugated to a payload that is toxic to the tumor-infiltrating Tregs. The present disclosure is not limited to any particular payload, but can incorporate any payload known in the art using standard conjugation techniques.

In some embodiments, the one or more agents comprise an engineered immune cell that expresses a bi-specific affinity reagent, as described above, or co-expresses a first chimeric antigen receptor (CAR) that specifically binds ICOS and a second chimeric antigen receptor (CAR) that specifically binds IL-1R1. The immune cell can be a T cell, an NK, or any other lymphocyte that can mediate toxicity in a target cell. In some embodiments, the engineered immune cell requires binding both ICOS and IL-1R1, e.g., by the first CAR and second CAR, to activate. Various approaches for functional CAR-expressing immune cells are known and are encompassed by embodiments of the present disclosure. See, e.g., Holzinger and Abken, CAR T Cells: A Snapshot on the Growing Options to Design a CAR, Hemasphere, 2019, 3(1):e172, incorporated herein by reference in its entirety. In other embodiments, the engineered immune cell expresses and secretes bi-specific antibodies that bind both ICOS and IL-1R1. The general approach is described in more detail in, e.g., Blanco, et al., Engineering Immune Cells for in vivo Secretion of Tumor-Specific T Cell-Redirecting Bispecific Antibodies, 2020, 13(11):11792 for different target antigens, incorporated herein by reference in its entirety.

In some embodiments, the engineered immune cell is a logic-gated CAR T cell that requires binding of the first CAR and the second CAR to induce a T cell response by the CAR T cell.

In a specific embodiment, the one or more agents comprise a logic-gated CAR T cell that co-expresses a first chimeric antigen receptor (CAR) that specifically binds one of ICOS and IL-1R1, and a second chimeric antigen receptor (CAR) that specifically binds to the other of ICOS and IL-1R1 via a bi-functional switch molecule. The CAR T cell requires binding by the first CAR and second CAR to induce a T cell response by the CAR T cell. In some further embodiments, the method further comprises contacting the solid tumor with an effective amount of the bi-functional switch molecule, wherein the bi-functional switch molecule comprises a first domain that specifically binds to the other of ICOS and IL-1R1 and a second domain that is specifically bound by the second CAR. An exemplary design of a gated dual receptor CAR T cell that incorporates a switch molecule is disclosed in Zhang et al., Accurate control of dual-receptor-engineered T cell activity through a bifunctional anti-angiogenic peptide, J Hematol Oncol. 2018; 11:44, incorporated herein by reference in its entirety.

In some embodiments, inhibiting or depleting the Tregs in the solid tumor reduces immunosuppressive conditions in the solid tumor.

The initial proof of concept that the ICOS+ and IL-1R1+ Treg cells are uniquely present in SSC solid tumors, as described below in Example 1, was performed in the context of SSC solid tumors. As described in Example 2, this unique expression pattern in tumor infiltrating Treg cells was also observed in breast cancer solid tumors. Accordingly, the present disclosure encompasses any solid tumor, for example SSC or breast cancer solid tumors.

In another aspect, the disclosure provides a method of treating a subject with a solid tumor, comprising administering to the subject a therapeutic composition comprising one or more agents that specifically bind inducible T cell costimulator (ICOS) and Interleukin-1 receptor type 1 (IL-1R1).

As used herein, the term “treat” refers to medical management of a disease, disorder, or condition (e.g., cancer such as SSC or breast cancer, as described herein) of a subject (e.g., a human or non-human mammal, such as another primate, horse, dog, mouse, rat, guinea pig, rabbit, and the like). Treatment can encompass any indicia of success in the treatment or amelioration of a disease or condition (e.g., a cancer), including any parameter such as abatement, remission, diminishing of symptoms or making the disease or condition more tolerable to the patient, slowing in the rate of degeneration or decline, or making the degeneration less debilitating. Specifically in the context of cancer, the term treat can encompass slowing, inhibiting, or reducing the rate of cancer growth, reducing cancer cell population or burden, or reducing the likelihood of recurrence, compared to not having the treatment. In some embodiments, the treatment encompasses resulting in some detectable degree of cancer cell death in the patient. The treatment or amelioration of symptoms can be based on objective or subjective parameters, including the results of an examination by a physician. Accordingly, the term “treating” includes the administration of the compositions of the present disclosure to alleviate, or to arrest or inhibit development of the symptoms or conditions associated with disease or condition (e.g., cancer). The term “therapeutic effect” refers to the amelioration, reduction, or elimination of the disease or condition, symptoms of the disease or condition, or side effects of the disease or condition in the subject. The term “therapeutically effective” refers to an amount of the composition that results in a therapeutic effect and can be readily determined

The one or more agents, and configurations thereof, are described in more detail above and are applicable to this aspect and are not repeated here. The therapeutic compositions can be formulated for any appropriate method or mode of administration for in vivo therapeutic settings in subjects (e.g., mammalian subjects with cancer). According to common knowledge and skill in the art, the disclosed therapeutic compositions can be formulated with appropriate carriers and non-active binders, and the like, for administration to target specific tumors and the Treg cells infiltrated therein. Because the compositions comprise binding domains that confer target cell specificity, the compositions can be formulated for direct or systemic administration according to skill and knowledge in the art.

The administration of the therapeutic composition can also be administered in combination with other therapeutic interventions, including other anti-cancer therapeutics. Any other cancer therapeutic strategy is contemplated in this combinatorial aspect. In some embodiments, the other cancer strategy is a cancer immunotherapy that utilizes immunomodulatory compositions (e.g., antibodies, immune cells, cytokines, etc.), which may boost the subject's own immune response against the cancer target. Such immune-therapies include adoptive immune cell therapies, including CAR T-cells, immune checkpoint inhibitor therapies, cancer vaccines, and the like. In certain embodiments, at least one additional therapeutic and the disclosed therapeutic composition are administered concurrently or in coordination to a subject. When administered in combination, each component can be administered at the same time or sequentially in any order at different points in time. Thus, each component can be administered separately but sufficiently closely in time so as to provide the desired therapeutic effect.

For example, the additional cancer therapy can comprise administration of a checkpoint inhibitor compound, an adoptive cell therapy, an anti-cancer antigen antibody or therapeutic composition.

Exemplary, non-limiting additional anti-cancer compositions can include cytotoxic agents that are known to further inhibit or treat the cancer. Nonlimiting examples include aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, duocarmycin, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (Taxol™), pilocarpine, prochloroperazine, rituximab, saproin, tamoxifen, taxol, topotecan hydrochloride, trastuzumab, vinblastine, vincristine, vinorelbine tartrate, and the like.

In some embodiments, the additional anti-cancer therapeutic is an immune checkpoint inhibitor. For example, current checkpoint inhibitors are known that inhibit PD-1, PD-L1, CTLA-4 LAG-3, Tim-3, or TIGIT. In some embodiments, the immune checkpoint inhibits PD-1, such as a checkpoint inhibitor selected from Pembrolizumab (Keytruda), Nivolumab (Opdivo), Cemiplimab (Libtayo), and the like. In some embodiments, the immune checkpoint inhibits PD-L1, such as a checkpoint inhibitor selected from Atezolizumab (Tecentriq), Avelumab (Bavencio), Durvalumab (Imfinzi), and the like. In some embodiments, the immune checkpoint inhibits CTLA-4, such as Ipilimumab (Yervoy), and the like.

In some embodiments, the additional anti-cancer therapeutic is a composition comprising immune cells for an adoptive cell therapy. Adoptive cell therapy is a technique by which cells, typically immune cells, are cultivated in vitro and administered to a subject to improve the immune functionality of the subject against a particular target. The immune cells can be autologous or allogenic. Exemplary immune cells include T cells and NK cells. In some embodiments, the immune cells are modified or enhanced by culture environments applied in vitro. In some embodiments, the immune cells are genetically modified to enhance or confer a new functionality. For example, the cells (e.g., T cells or NK cells) can be genetically modified to express a chimeric antigen receptor (CAR) on the surface. The CAR typically contains an extracellular domain with enhanced affinity for an antigen of interest. The extracellular domain is linked to an intracellular signaling domain that activates the cell upon antigen binding. Such CAR-expressing cells can provide a powerful tool to combat cancer cells because upon binding to the target antigen in vivo, the CAR-expressing cells undergo further expansion and activation to provide a type of “living drug” that can have a direct cytotoxic action against the target as well as influence the endogenous immune functionality through production of cytokines. In some embodiments, when the CAR-expressing immune cell is an additional composition combined with the disclosed therapeutic composition, the expressed CAR can be specific for a tumor cell-associated antigen.

In some embodiments, the solid tumor is a squamous cell carcinoma (SCC) or a breast cancer tumor.

In another aspect, the disclosure provides a composition comprising an engineered immune cell that co-expresses a first chimeric antigen receptor (CAR) that specifically binds ICOS and a second chimeric antigen receptor (CAR) that specifically binds IL-1R1. The engineered immune cell requires binding by the first receptor and second receptor to activate. The immune cell can be a T cell, NK cell, or other lymphocyte. The engineered immune cell can be a logic-gated CAR T cell that requires binding of the first CAR and the second CAR to induce a T cell response by the CAR T cell.

In a related aspect, the disclosure provides a composition comprising a logic-gated CAR T cell that co-expresses a first chimeric antigen receptor (CAR) that specifically binds one of ICOS and IL-1R1, and a second chimeric antigen receptor (CAR) that specifically binds to the other of ICOS and IL-1R1 via a bi-functional switch molecule. The CAR T cell requires binding by the first CAR and second CAR to induce a T cell response by the CAR T cell. The bi-functional switch molecule can comprise a first domain that specifically binds to the other of ICOS and IL-1R1 and a second domain that is specifically bound by the second CAR, and the CAR T cell requires simultaneous binding by the first domain to the other of ICOS and IL-1R1 and the second domain to the second CAR to induce a T cell response by the CAR T cell.

In another aspect, the disclosure provides a composition comprising one or more agents that specifically bind ICOS and IL-1R1 on Treg cells. For example, the one or more one or more agents can be a single bi-specific affinity reagent that binds to both ICOS and IL-1R1. Alternatively, the one or more agents can be two distinct mono-specific reagents that bind to ICOS and IL-1R1, respectively. Exemplary structures of the affinity reagents encompassed by this aspect are described above in more detail. In this aspect, the affinity reagent(s) is/are conjugated to a payload that is toxic to the tumor-infiltrating Tregs. The present disclosure is not limited to any particular payload, but can incorporate any payload known in the art using standard conjugation techniques.

Additional features of CAR-expressing immune cells, including logic gated dual receptor expressing immune cells are described in more detail above and are encompassed by these aspects. The composition can be formulated for any appropriate mode of administration, such as systemic administration, with the appropriate carriers, etc.

In another aspect, the disclosure provides methods of detecting tumor-infiltrating Treg cells. In one embodiment, a tumor is contacted in vivo by one or more agents that specifically bind inducible T cell costimulator (ICOS) and Interleukin-1 receptor type 1 (IL-1R1), wherein the one or more agents are detectably labeled. A tumor biopsy is then extracted and assessed for binding of the one or more agents to ICOS and IL-1R1. In another embodiment, the method comprises contacting a sample comprising tumor cells obtained from a subject with a solid tumor with one or more agents that specifically bind ICOS and IL-1R1, wherein the one or more agents are detectably labeled. The method further comprises detecting binding of the one or more agents to a cell in the sample, wherein binding the one or more agents to a cell in the sample indicates the presence of tumor-infiltrating Treg cells in the tumor environment in the subject.

The one or more agents can be one or multiple of the affinity reagents, described in more detail above. Exemplary, non-limiting categories of affinity reagent include antibodies, an antibody-like molecule (including antigen-binding fragments of antibody fragments, derivatives), peptides that specifically interact with a particular antigen (e.g., peptibodies), antigen-binding scaffolds (e.g., DARPins, HEAT repeat proteins, ARM repeat proteins, tetratricopeptide repeat proteins, and other scaffolds based on naturally occurring repeat proteins, etc., [see, e.g., Boersma and Pluckthun, Curr. Opin. Biotechnol. 22:849-857, 2011, and references cited therein, each incorporated herein by reference in its entirety]), aptamers, or a functional ICOS and IL-1R1-binding domain or fragment thereof

In some embodiments, the one or more agents comprise a bi-specific affinity reagent with a first domain that specifically binds ICOS and a second domain that specifically binds IL-1R1. In another embodiment, the one or more agents comprises a first affinity reagent that specifically binds ICOS and a second affinity reagent that specifically binds IL-1R1.

The detectable labels can be any detectable label known and used in the art. In some embodiments, the first affinity reagent produces a first detectable signal and the second affinity reagent produces a second affinity signal that is different from the first detectable signal.

In some embodiments, the detecting binding of the one or more agents to a cell in the sample comprises flow cytometry. In some embodiments, the detecting binding of the one or more agents to a cell in the sample comprises an immune assay.

In some embodiments, the method further comprises treating the subject with a determined presence of tumor-infiltrating Treg cells in the tumor environment with a treatment to inhibit or deplete the tumor-infiltrating Treg cells. An exemplary treatment for purposes of this aspect is described above, but the treatment can encompass any method of treatment that inhibits or depletes Treg cells in the tumor environment.

### Additional Definitions

Unless specifically defined herein, all terms used herein have the same meaning as they would to one skilled in the art of the present invention. Practitioners are particularly directed to Sambrook J., et al. (eds.), Molecular Cloning: A Laboratory Manual, 3rd ed., Cold Spring Harbor Press, Plainsview, New York (2001); Ausubel, F. M., et al. (eds.), Current Protocols in Molecular Biology, John Wiley & Sons, New York (2010); and Coligan, J. E., et al. (eds.), Current Protocols in Immunology, John Wiley & Sons, New York (2010) for definitions and terms of art.

The use of the term “or” in the claims is used to mean “and/or” unless explicitly indicated to refer to alternatives only or the alternatives are mutually exclusive, although the disclosure supports a definition that refers to only alternatives and “and/or.”

Following long-standing patent law, the words “a” and “an,” when used in conjunction with the word “comprising” in the claims or specification, denotes one or more, unless specifically noted.

Unless the context clearly requires otherwise, throughout the description and the claims, the words “comprise,” “comprising,” and the like, are to be construed in an inclusive sense as opposed to an exclusive or exhaustive sense; that is to indicate, in the sense of “including, but not limited to.” Words using the singular or plural number also include the plural and singular number, respectively. Additionally, the words “herein,” “above,” and “below,” and words of similar import, when used in this application, shall refer to this application as a whole and not to any particular portions of the application. The word “about” indicates a number within range of minor variation above or below the stated reference number. For example, “about” can refer to a number within a range of 10%, 9%, 8%, 7%, 6%, 5%, 4%, 3%, 2%, or 1% above or below the indicated reference number.

The terms “subject,” “individual,” and “patient” are used interchangeably herein to refer to a mammal being assessed for treatment and/or being treated. In certain embodiments, the mammal is a human. The terms “subject,” “individual,” and “patient” encompass, without limitation, individuals having cancer. While subjects may be human, the term also encompasses other mammals, particularly those mammals useful as laboratory models for human disease, e.g., mouse, rat, dog, non-human primate, and the like.

The term “treating” and grammatical variants thereof may refer to any indicia of success in the treatment or amelioration or prevention of a disease or condition (e.g., a cancer, infectious disease, or autoimmune disease), including any objective or subjective parameter such as abatement; remission; diminishing of symptoms or making the disease condition more tolerable to the patient; slowing in the rate of degeneration or decline; or making the final point of degeneration less debilitating.

The treatment or amelioration of symptoms can be based on objective or subjective parameters; including the results of an examination by a physician. Accordingly, the term “treating” includes the administration of the compounds or agents of the present disclosure to prevent or delay, to alleviate, or to arrest or inhibit development of the symptoms or conditions associated with disease or condition (e.g., a cancer, infectious disease, or autoimmune disease). The term “therapeutic effect” refers to the reduction, elimination, or prevention of the disease or condition, symptoms of the disease or condition, or side effects of the disease or condition in the subject.

As used herein, characterization of a cell or population of cells being “positive” (or “+”) for a particular marker (or markers) refers to the cell or population of cells having the detectable presence of the marker(s). Often, the marker is present or expressed on the surface of the cell. The marker can be detected using any conventional techniques. To detect the surface expression, for example, the marker can be detected using immune-staining based techniques. For example, an antibody specific for the marker can be exposed to the cell or population of cells and the binding of the antibody can be imaged or detected by flow cytometry. Conversely, use of the term “negative” (or “−”) refers to the absence of a substantial presence in or on the surface of the cell.

Disclosed are materials, compositions, and components that can be used for, can be used in conjunction with, can be used in preparation for, or are products of the disclosed methods and compositions. It is understood that, when combinations, subsets, interactions, groups, etc., of these materials are disclosed, each of various individual and collective combinations is specifically contemplated, even though specific reference to each and every single combination and permutation of these compounds may not be explicitly disclosed. This concept applies to all aspects of this disclosure including, but not limited to, steps in the described methods. Thus, specific elements of any foregoing embodiments can be combined or substituted for elements in other embodiments. For example, if there are a variety of additional steps that can be performed, it is understood that each of these additional steps can be performed with any specific method steps or combination of method steps of the disclosed methods, and that each such combination or subset of combinations is specifically contemplated and should be considered disclosed. Additionally, it is understood that the embodiments described herein can be implemented using any suitable material such as those described elsewhere herein or as known in the art.

Publications cited herein and the subject matter for which they are cited are hereby specifically incorporated by reference in their entireties.

### EXAMPLES

The following examples are set forth so as to provide those of ordinary skill in the art with a complete disclosure and description of how to make and use the present invention, and are not intended to limit the scope of what the inventors regard as their invention nor are they intended to represent that the experiments below are all or the only experiments performed.

### Example 1

### Introduction

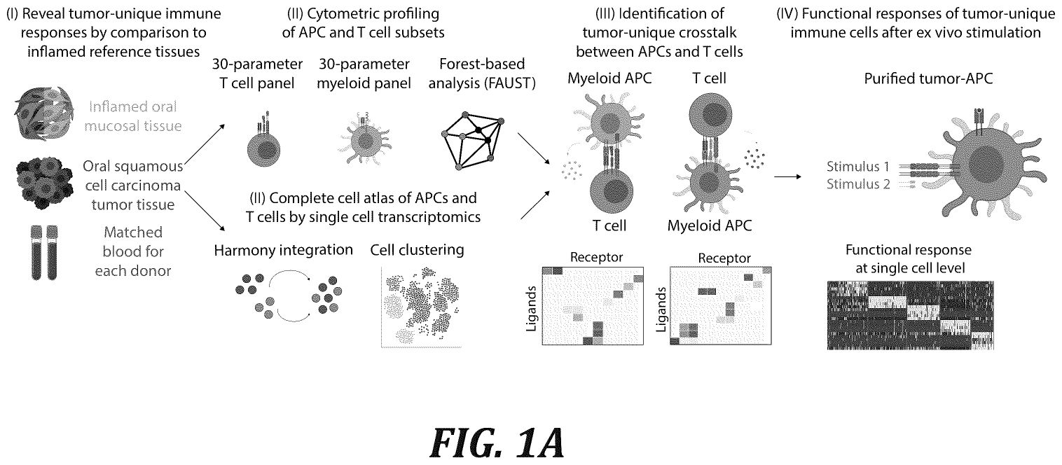

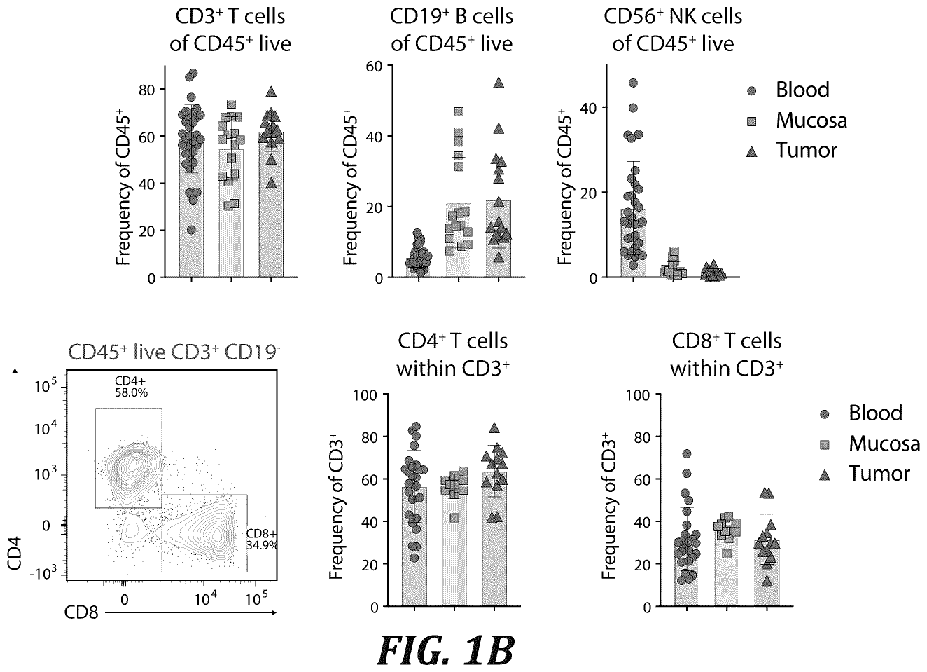

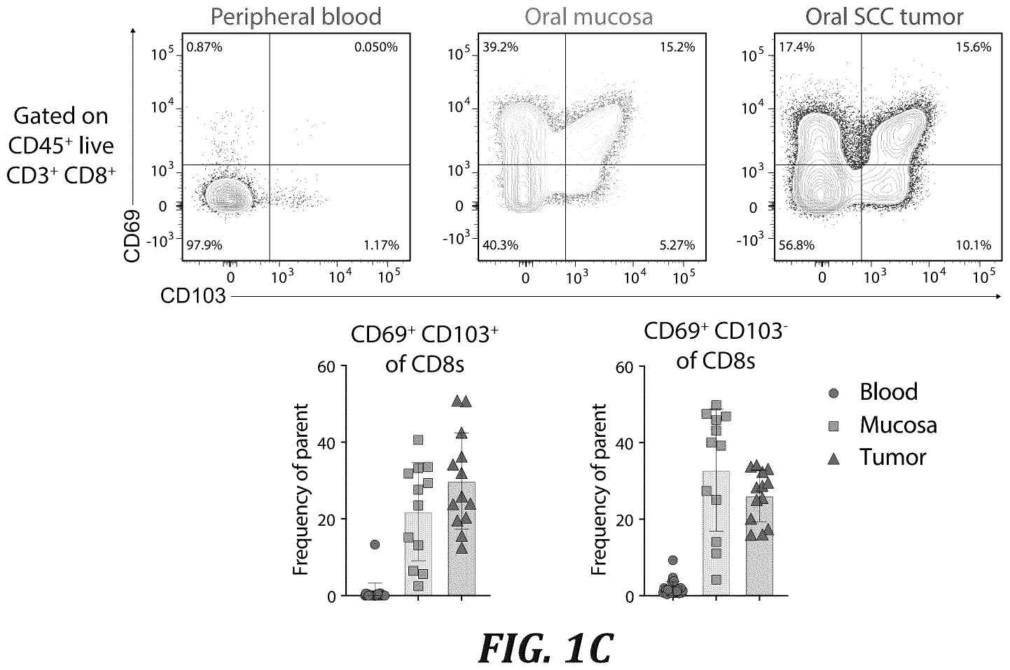

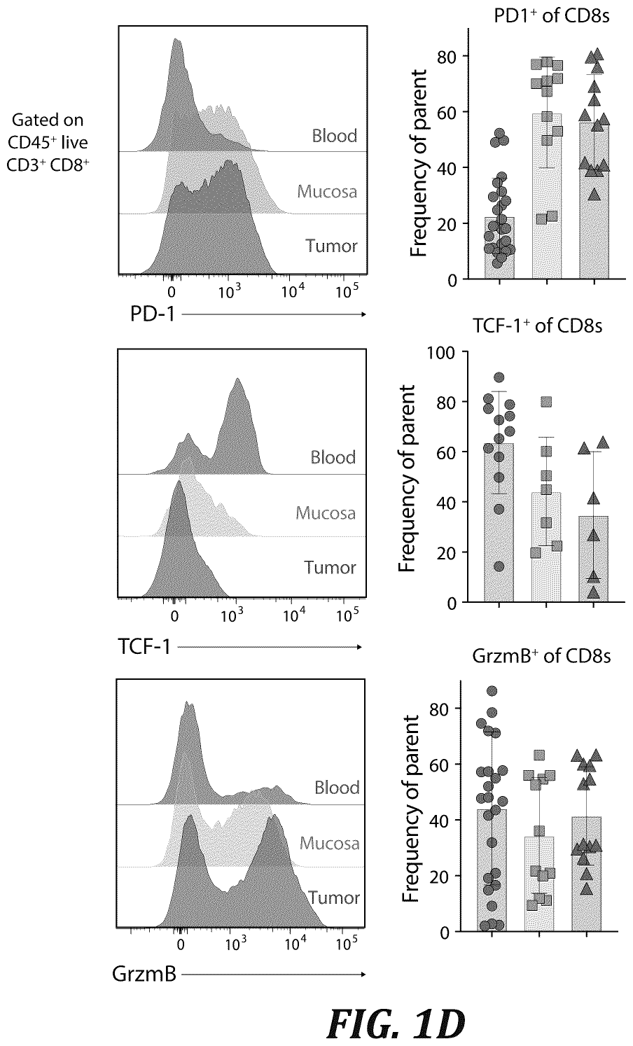

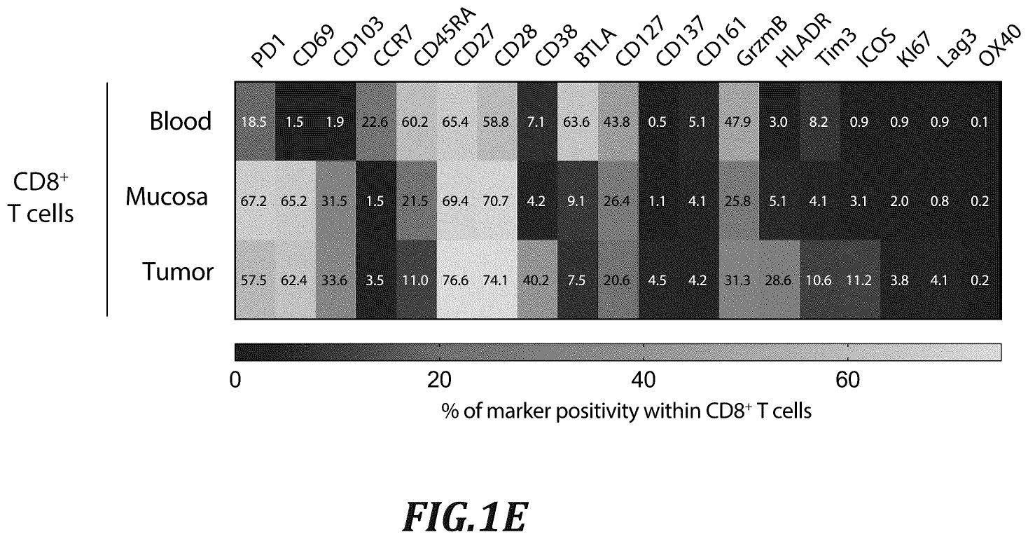

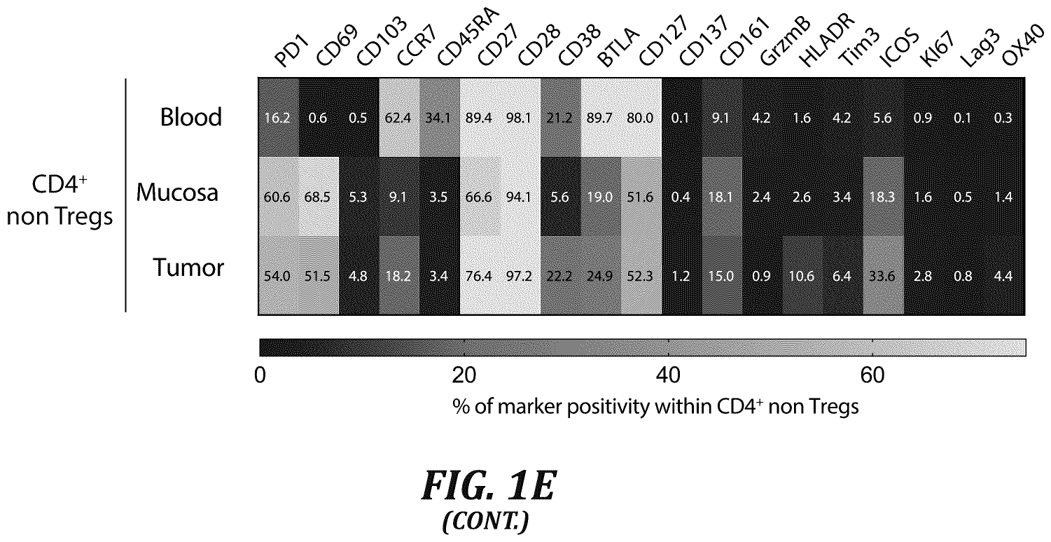

This Example discloses generation of a comprehensive single cell atlas of myeloid APCs and T cells in oral squamous cell carcinoma (SCC) biopsies relative to inflamed oral tissue biopsies from otherwise healthy individuals (experimental overview provided in FIG. 1A). For this purpose, SCC represents a well-suited tumor type because of the limited treatment options (primarily surgical resection and radiation therapy), and also the fact that most patients undergoing surgery are treatment naïve, i.e. allowing to study the endogenous immune response without influence from prior therapeutic intervention. Using high-parameter cytometric profiling and a novel computational method for unbiased cell population discovery, FAUST (Full Annotation Using Shape-constrained Trees), CD8⁺ T cells were found to have comparable expression patterns of several exhaustion markers between tumor and inflamed tissue samples, including PD-1. Contrarily, one major immune phenotype showing unique enrichment in tumor biopsies was a specific subset of Tregs with tissue-resident profile co-expressing CD69, PD-1, ICOS and HLA-DR.

NicheNet analysis of APC-T cell crosstalk using this comprehensive sc-RNAseq data on more than 150,000 cells from multiple donors revealed that signals from IL-1, IL-18 and ICOS ligand were integrated by the Treg compartment only. Validation experiments confirmed that surface protein expression of the IL-1 receptor type 1 (IL-1R1) was exclusively restricted to tumor-infiltrating ICOS⁺ HLA-DR⁺ Tregs, and that IL-1R1 in conjunction with ICOS can be used as biomarkers to specifically target tumor-infiltrating Tregs.

This work establishes a novel workflow for the identification of specific tumor-unique immune adaptation, and can serve as a reference data set and blueprint for revealing more specific therapeutic targets in other human tumor types. More particularly, this work established a specific expression profile of tumor-infiltrating Treg cells, providing a unique target for therapy that can avoid deleterious effects of general Treg depletion.

Results

The CD4⁺ and CD8⁺ T Cell Phenotypes in SCC Show Large Phenotypic Overlap with Inflamed Reference Tissues

A total of 25 matched peripheral blood samples and tissue biopsies were collected from the oral cavity either from patients undergoing routine dental surgeries with different degrees of inflammation, or from surgical resections of oral squamous cell carcinoma (SCC) lesions. To obtain a comprehensive snapshot of the tissue-infiltrating immune subsets, two 30-parameter flow cytometry panels focused on T cells and myeloid antigen-presenting cells (APCs) were developed (FIG. 1A). The frequency of CD3⁺ T cells, CD19⁺ B cells and CD56⁺ NK cells among total CD45⁺ live cells as well as the CD4/CD8 ratio was undistinguishable between mucosal tissues and SCC tissues (FIG. 1B). Of note, compared to peripheral blood, in both tissue types the relative B cell abundance was increased, with a parallel decrease in CD56⁺ NK cells.

Given that CD8⁺ cytotoxic T cells with a tissue-resident memory phenotype have been suggested to be a principal predictor for tumor progression, the expression of the tissue residency markers CD69 and CD103 was assessed, which was very similar between mucosal and SCC tissues (FIG. 1C). Importantly, programmed death 1 (PD-1), which has been suggested to be a defining molecule of exhausted T cells in the tumor microenvironment, was found on approximately 50% of total CD8⁺ T cells both in mucosal and SCC tissue samples (FIG. 1D). Furthermore, expression of the effector molecule Granzyme B and the transcription factor TCF-1 was assessed, which have been suggested to be critical for the development of terminally exhausted CD8⁺ T cell subsets, also revealing comparable expression patterns (FIG. 1D).

When the overall phenotype across all markers for CD8⁺ cytotoxic T cells as well as CD4⁺ helper T cells on a heatmap was evaluated, it was found that compared to peripheral blood both mucosal as well as SCC tissues showed similar changes in the expression of several key markers, such as downregulation of CD45RA and the chemokine receptor CCR7 as well as the IL-7 receptor (CD127). Of note, in line with previous reports, slightly increased expression of CD38 was observed in tumor-infiltrating CD4⁺ helper T cells and CD8⁺ T cells, as well as a consistent up-regulation of HLA-DR (FIG. 1E).

Computational Analysis Using FAUST Reveals a Tumor-Specific Treg Phenotype Co-Expressing HLA-DR and ICOS

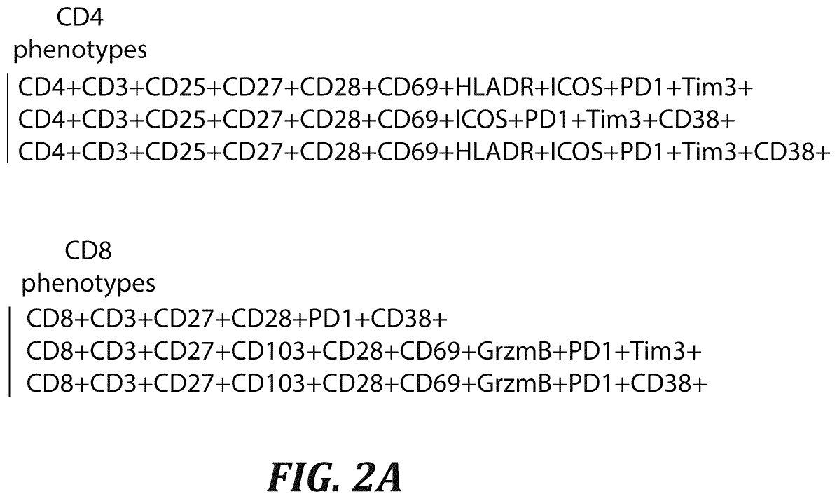

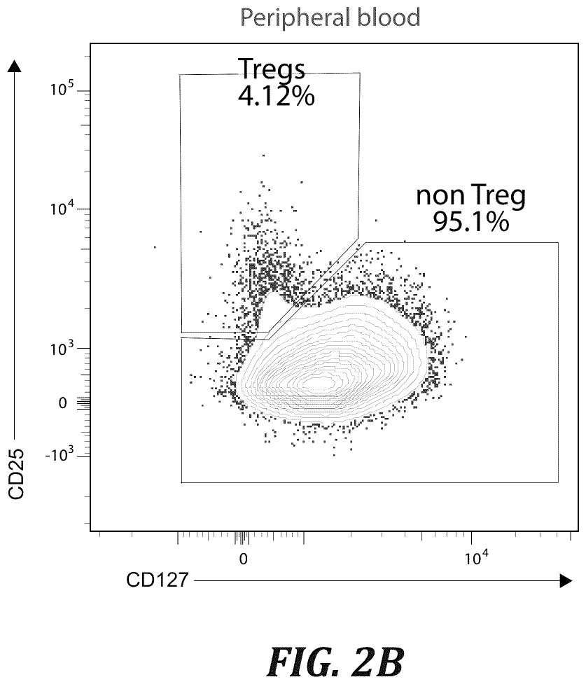

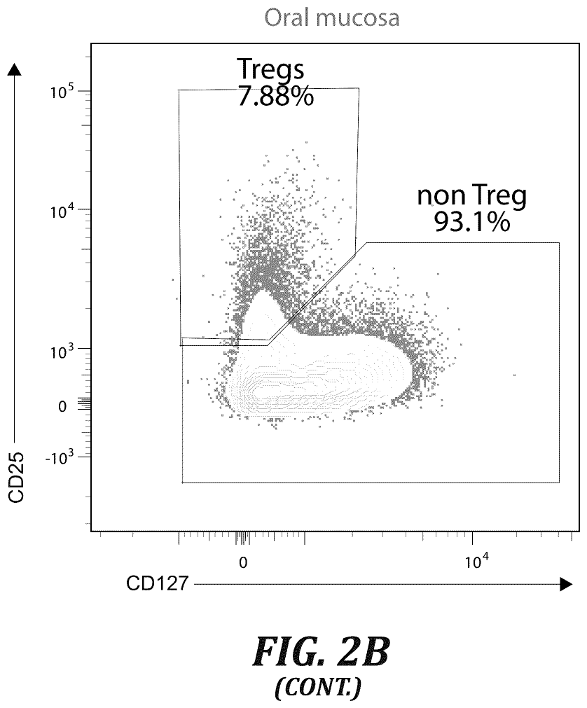

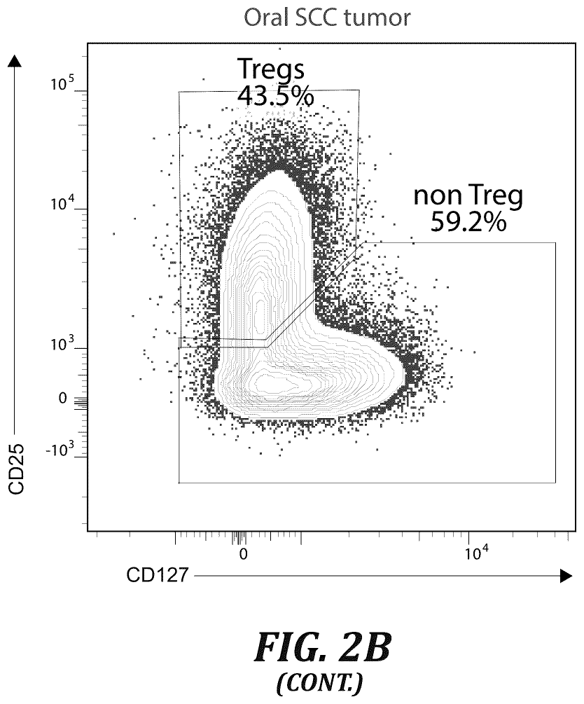

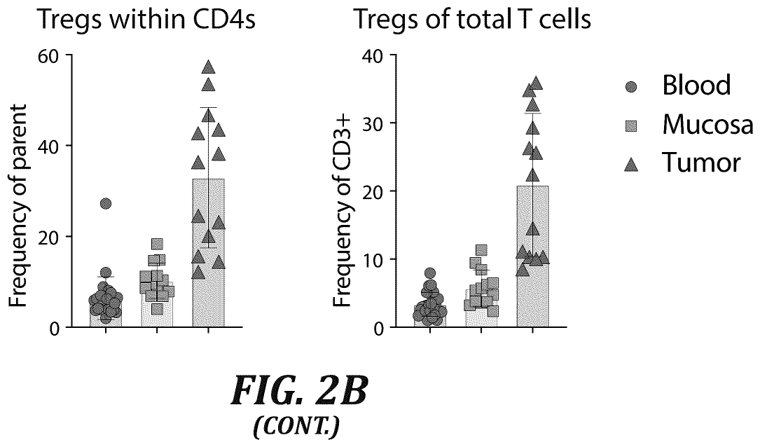

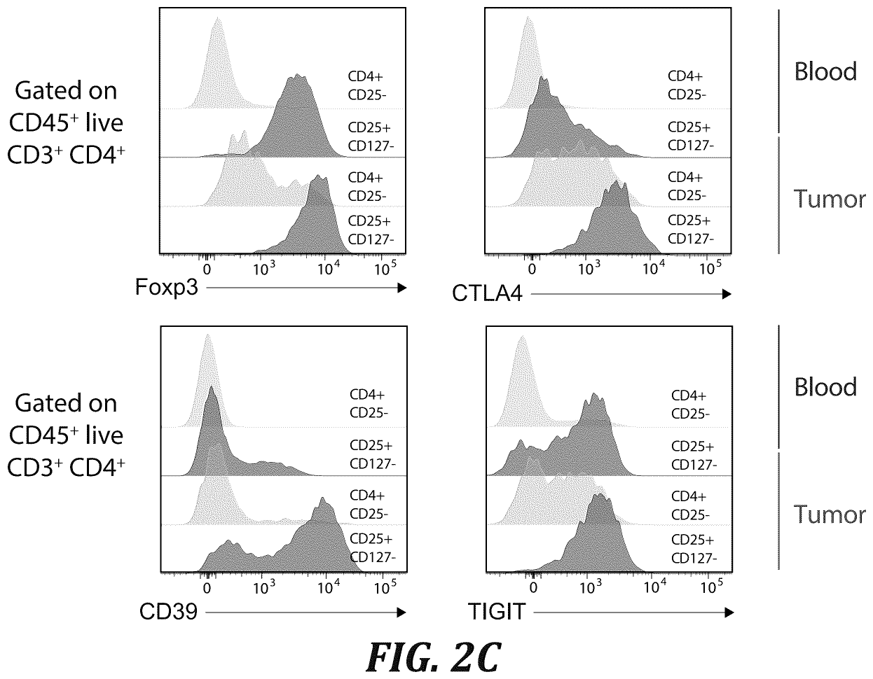

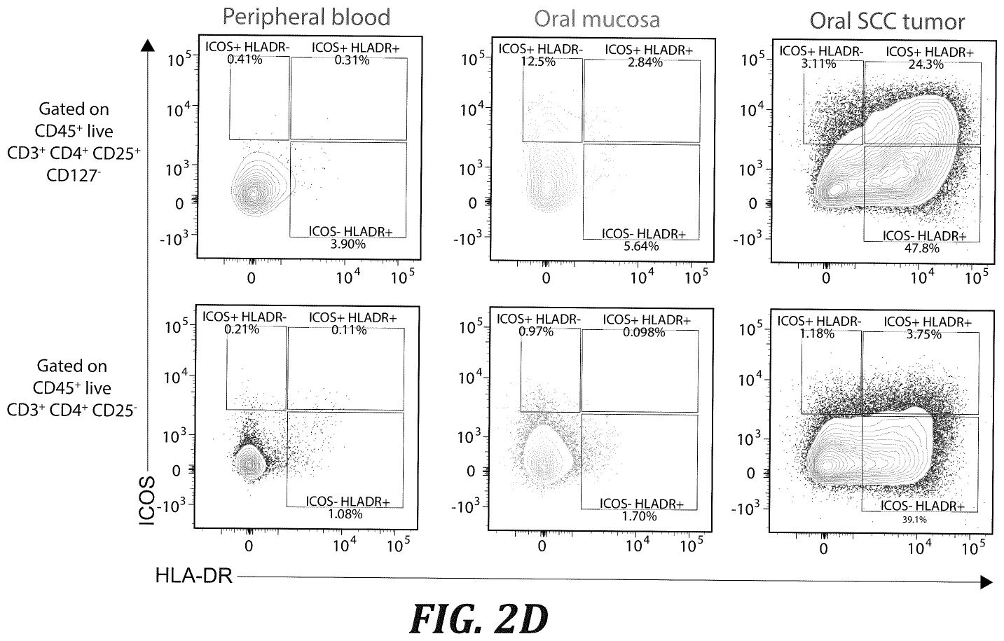

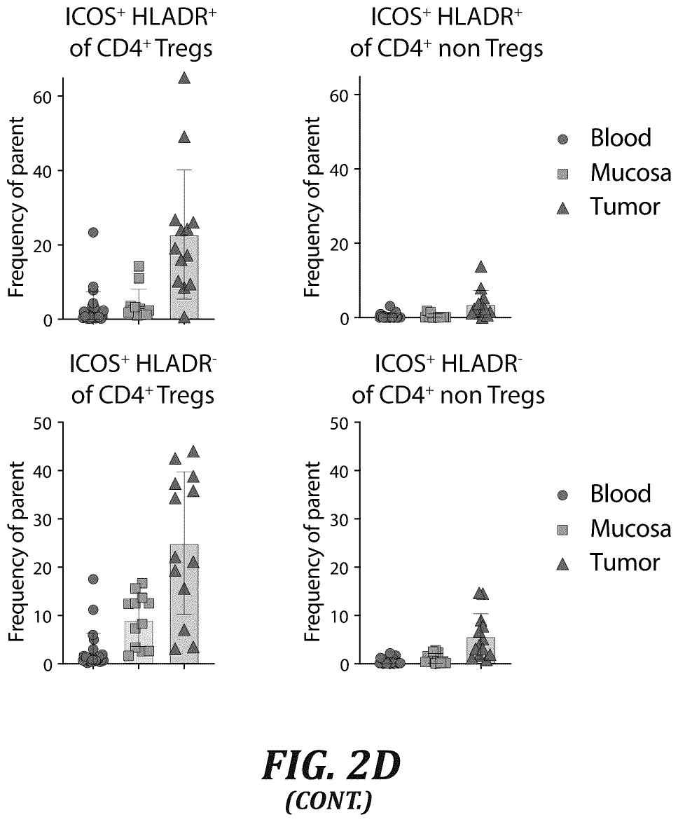

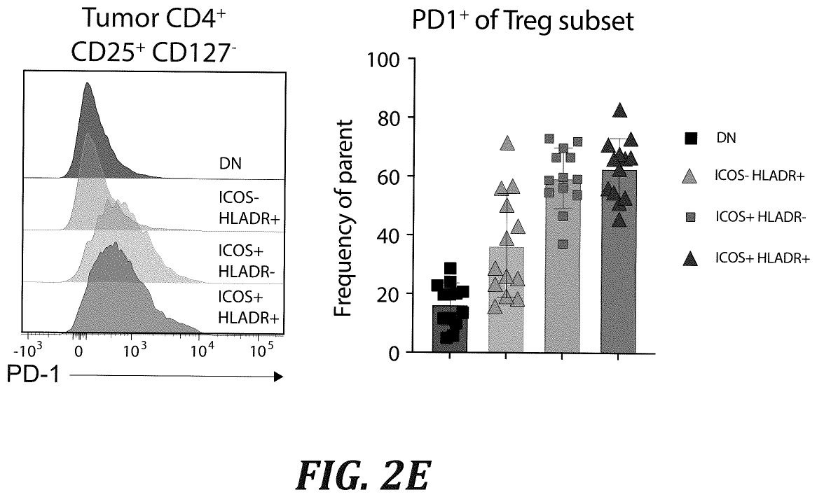

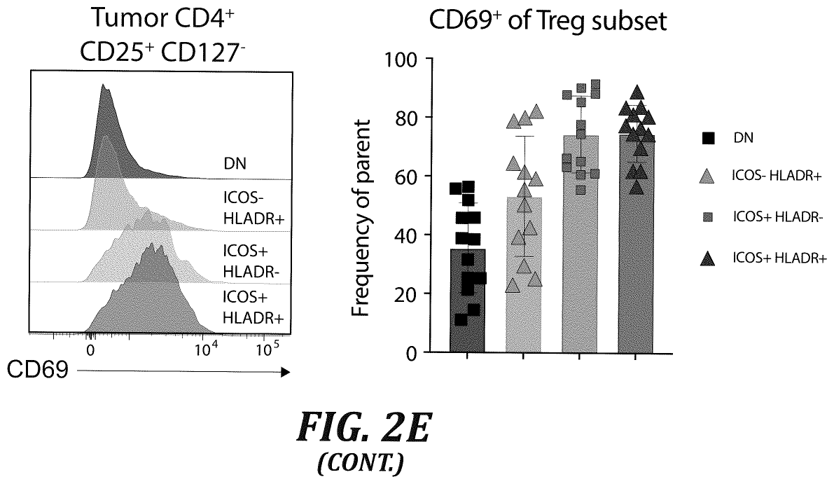

Based on the large congruence between the tissue T cell phenotypes as identified by manual gating, an exploratory computational analysis was performed of the cytometry data using Full Annotation Using Shape-constrained Trees (FAUST). Briefly, FAUST performs data-driven automated gating on a per-sample basis and identifies condition-specific phenotypes (in this case, tumor relative to mucosa) using a multivariate modeling framework called phenotypic and functional differential abundance (PFDA). The top phenotypes identified by FAUST as enriched or unique to the tumor microenvironment fell into two distinct categories (FIG. 2A): First, CD8⁺ CD27⁺ CD28⁺ T cells co-expressing different combinations of the tissue residency markers CD69, CD103 and the activation/exhaustion markers PD-1, Tim3 as well as CD38, corroborating the emerging consensus that PD-1 alone might be insufficient as a marker for tumor-specific cytotoxic CD8⁺ T cells.

The second category of highest-scoring phenotypes identified by FAUST were three different subsets of CD4⁺ CD27⁺ CD28⁺ CD25⁺ CD127⁻ regulatory T cells (Tregs) expressing the tissue residency marker CD69, as well as PD-1, Tim3, inducible T cell costimulatory (ICOS) and HLA-DR. When the overall abundance of Tregs was assessed, up to 50% of CD4⁺ T cells in the tumor were CD25⁺ CD127⁻ Tregs, in line with published work and increased about 5-fold relative to inflamed mucosal tissue samples (FIG. 2B). Using a different panel focused on transcriptions factors these cells were confirmed to express Foxp3, as well as high levels of CTLA-4, CD39 and T cell immunoreceptor with Ig and ITIM domains (TIGIT), suggesting that CD25⁺ CD127⁻ CD4⁺ cells in the tumor are indeed bona-fide suppressive Tregs (FIG. 2C).

When the computationally predicted phenotypes were simplified for manual gating, it was observed that cells co-expressing ICOS and HLA-DR were enriched primarily in the tumor-infiltrating Treg fraction, but neither in CD25⁻ CD4⁺ conventional helper T cells (FIG. 2D) nor CD8⁺ cytotoxic T cells. Of the ICOS⁺ HLA-DR⁺ cells, almost all cells expressed intermediate levels of PD-1 as well as positivity for CD69 (FIG. 2E), in line with the FAUST analysis and suggesting a tissue-resident Treg program. In summary, this computational analysis revealed that compared to a generally inflamed tissue microenvironment, a novel and specific T cell phenotype found in solid human SCC tumor tissue comprises ICOS⁺ HLA-DR⁺ PD1⁺ CD69⁺ Tregs.

The APC Compartment in the SCC Microenvironment Shows Large Phenotypic Heterogeneity and an Activated cDC2 Phenotype

The next aim was to elucidate which factors could be driving the unique T cell phenotypes that were identified in SCC tissue. Professional APCs, including DCs and macrophages have been shown to be critical players for steering T cell activation and function, and in particular cross-presenting cDC1s have been identified as a cellular population predictive for tumor outcome. However, a number of previous studies on myeloid cells relied on single markers for identification of canonical subsets, as well as single markers for polarization of myeloid cells, such as CD206 for so-called M2 macrophages. These simplistic notions have been challenged by a large body of recent work highlighting the heterogeneity and diversity of the mononuclear phagocyte system. Particular during inflammatory conditions in tissues, the classical distinction between monocytes and dendritic cells has become blurred, with the recent description of an inflammatory DC3 phenotype in the peripheral blood as well as tumors, emphasizing the importance for deep phenotyping of myeloid cells.

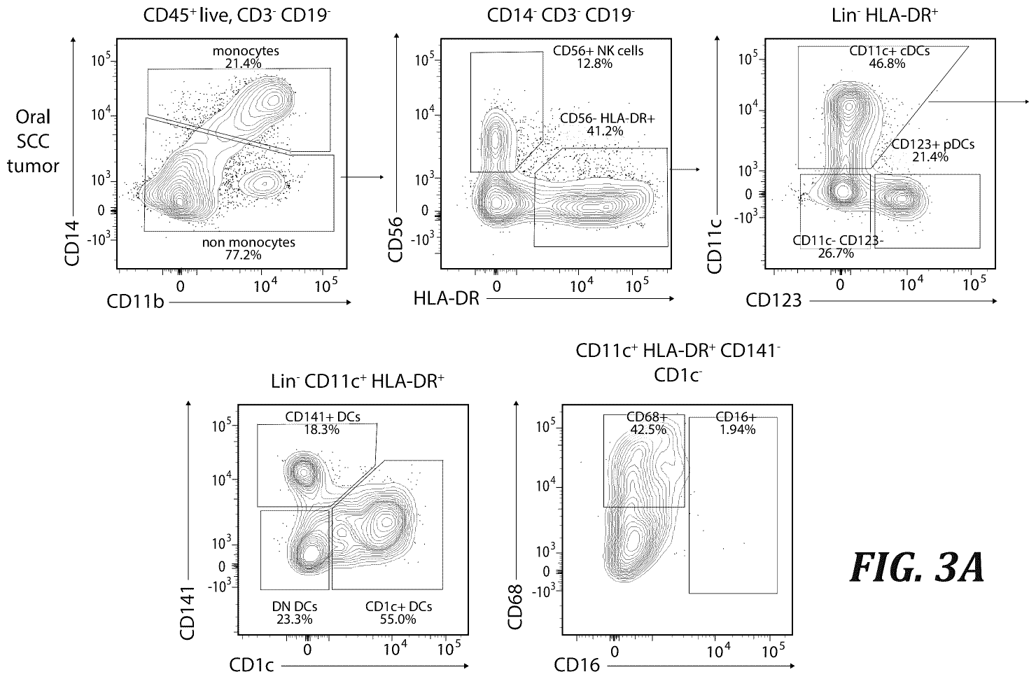

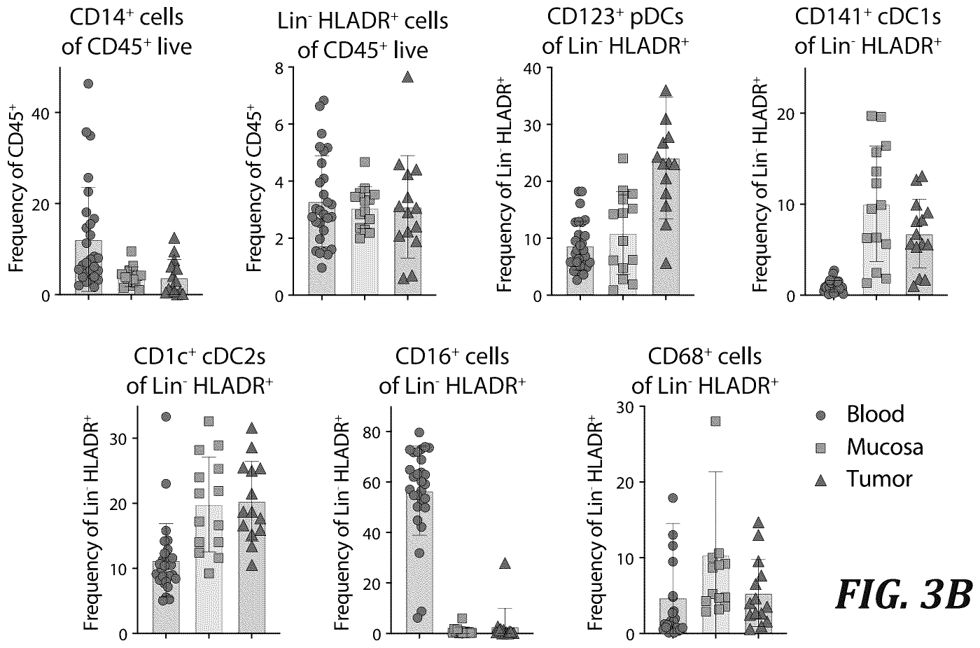

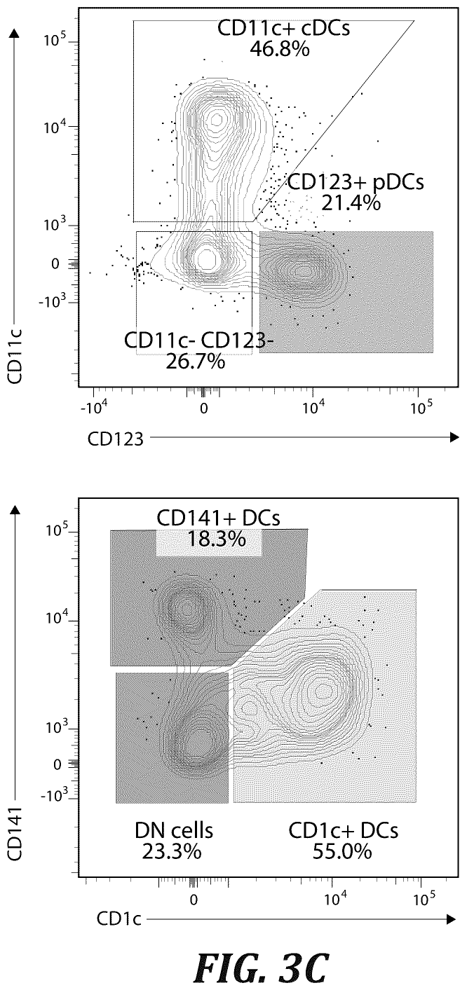

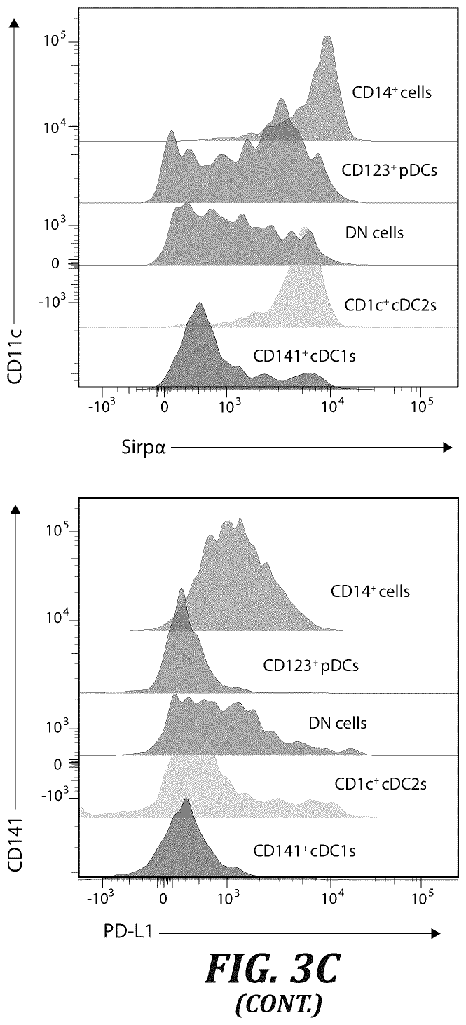

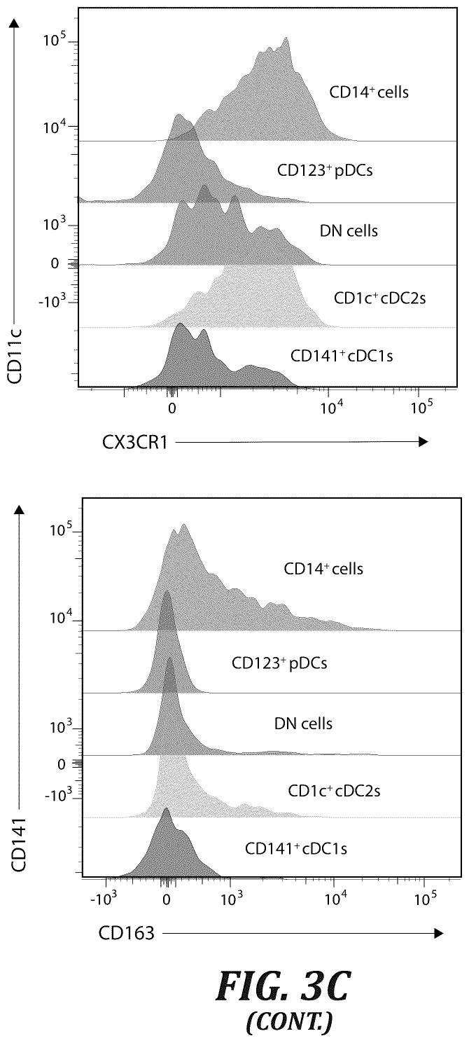

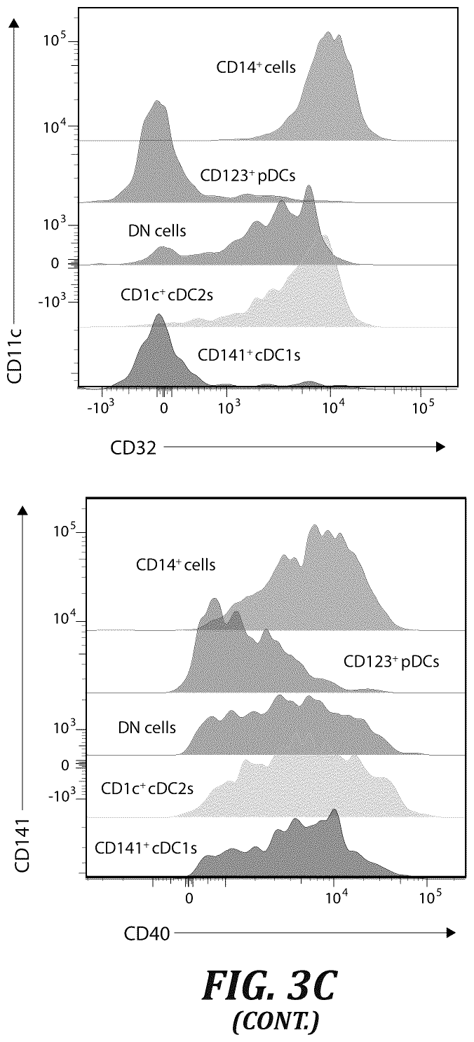

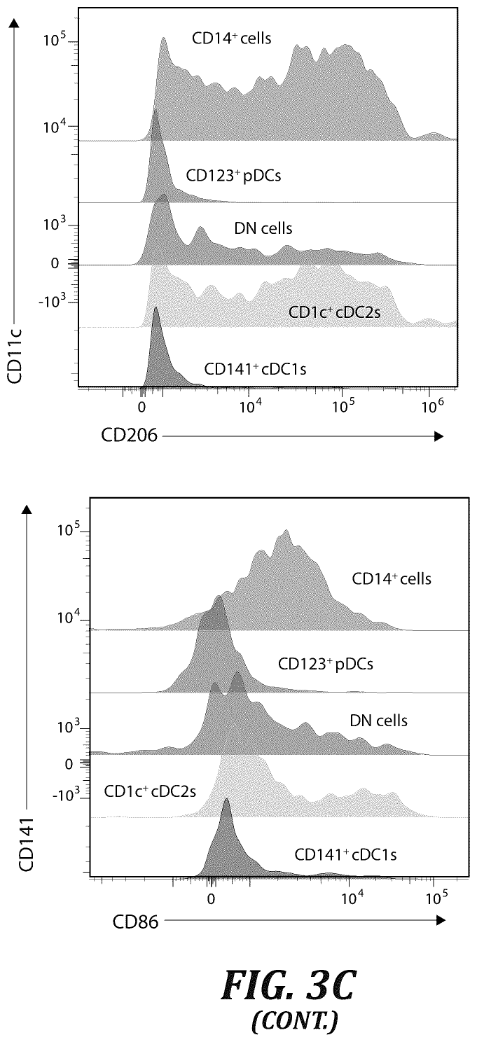

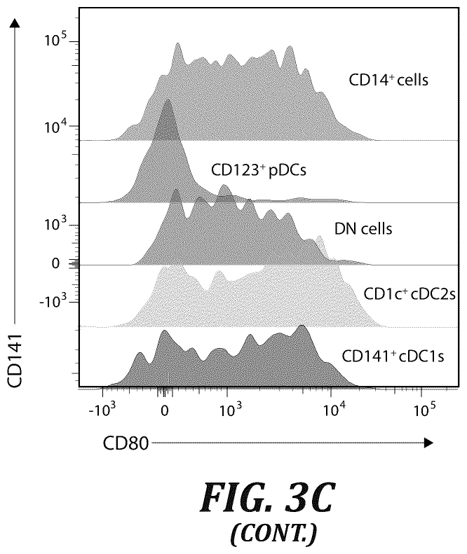

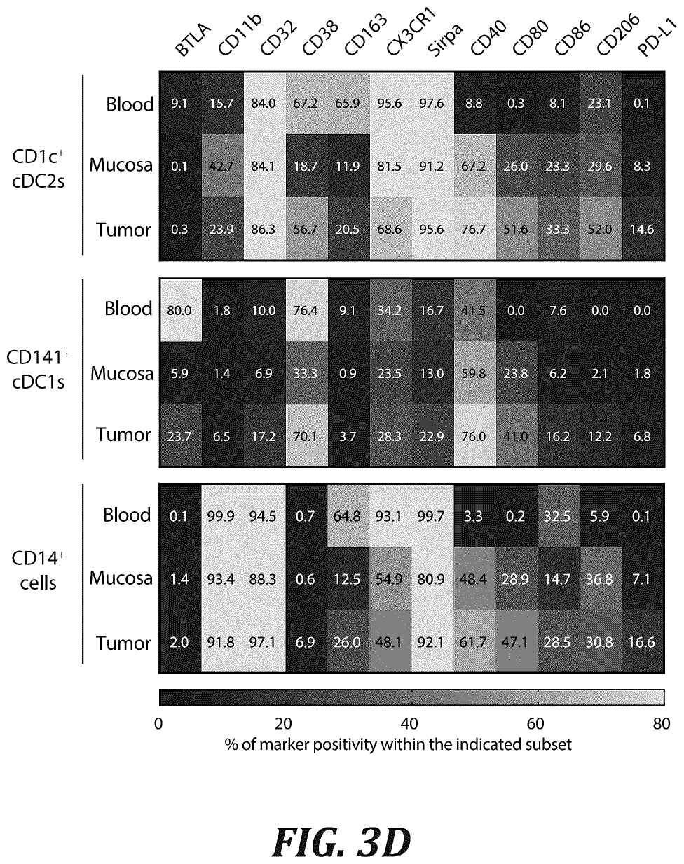

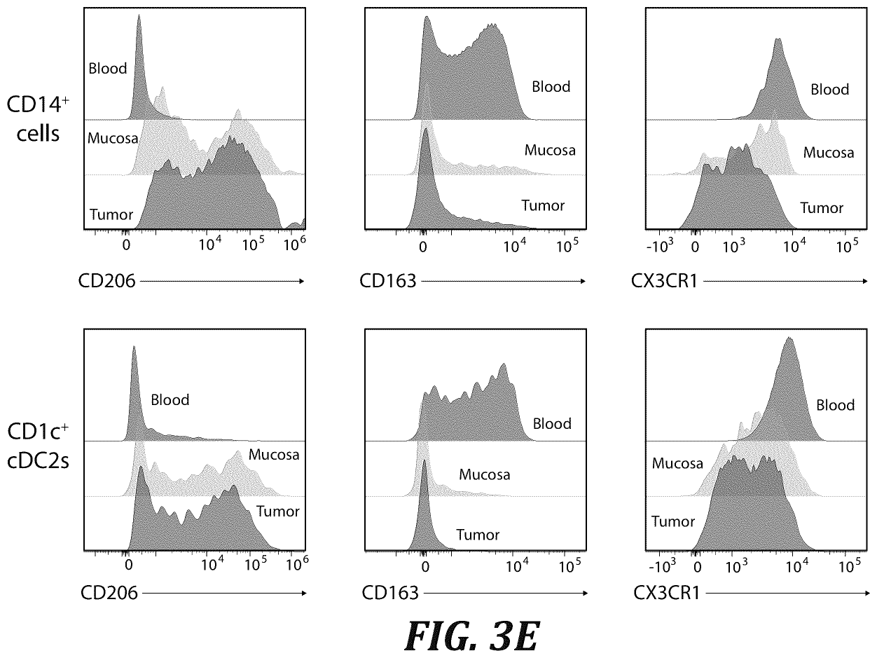

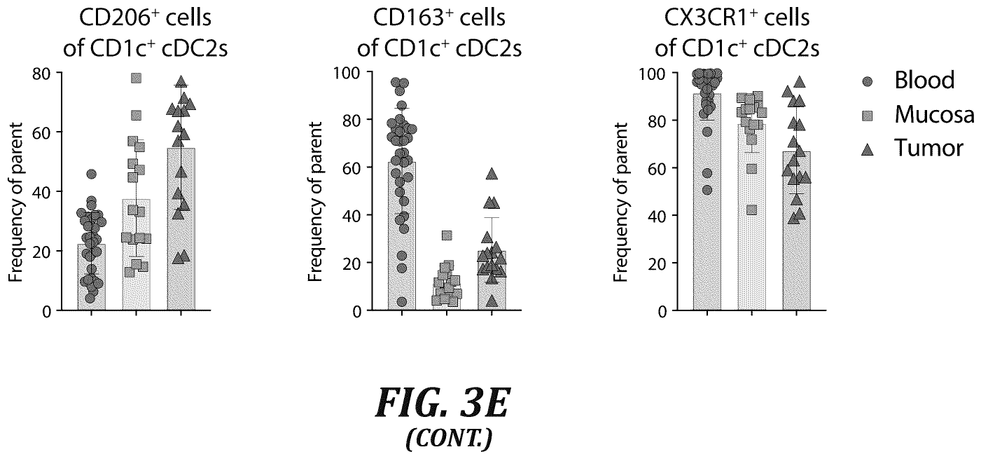

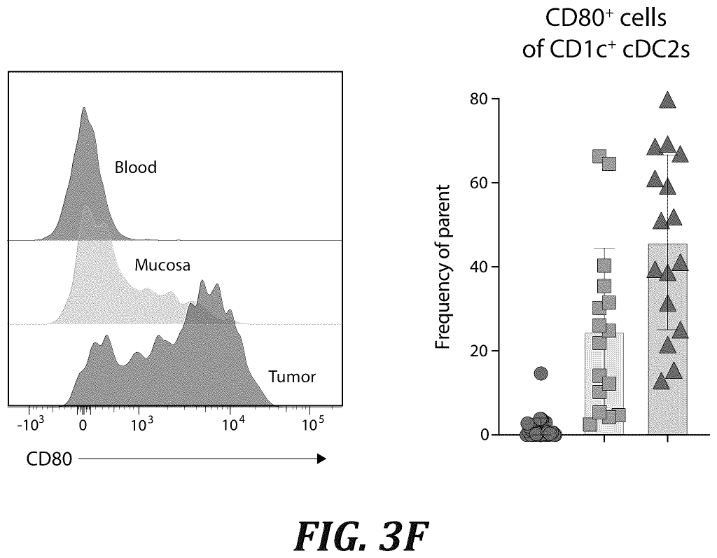

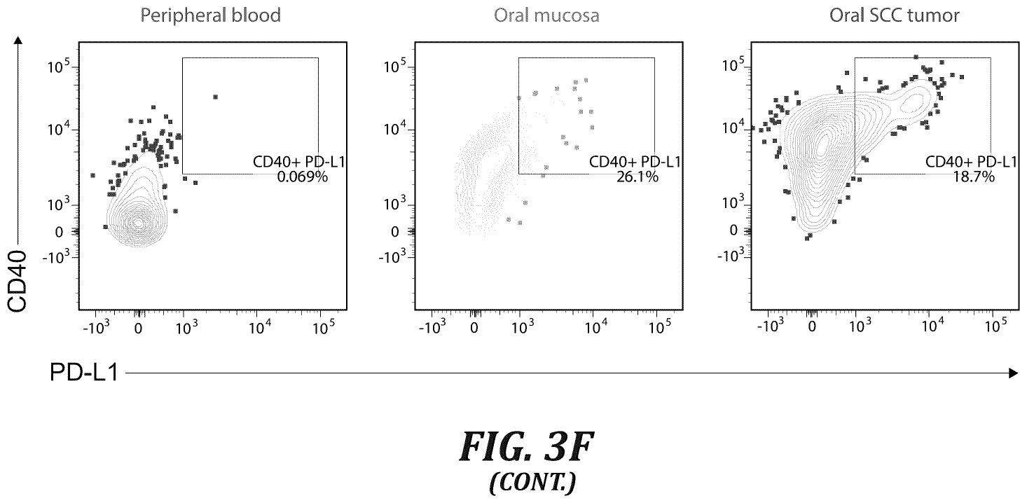

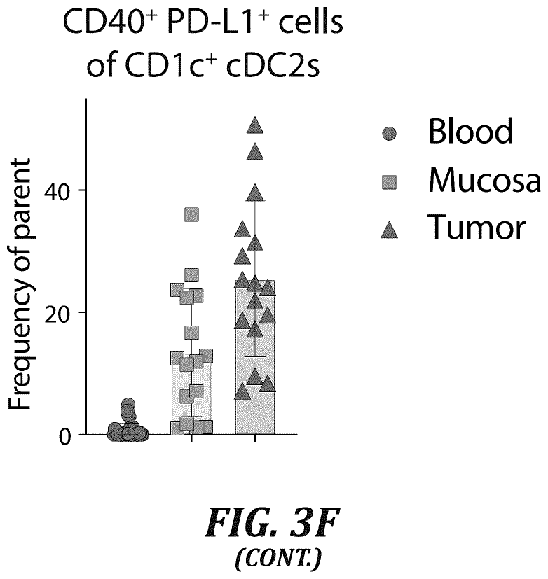

First, subsets of monocytes and dendritic cells were assessed in the tumor microenvironment based on canonical lineage markers, identifying CD14⁺ monocyte/macrophage-like cells, and within the CD14⁻ CD3⁻ CD19⁻ CD56⁻ (lineage-) HLA-DR⁺ fraction CD123⁺ plasmacytoid DCs, CD11c⁺ CD141⁺ cross-presenting cDC1s, CD11c⁺ CD1c cDC2s, and within the CD141⁻ CD1c⁻ negative fraction CD68⁺ cells and CD16⁺ cells (FIG. 3A).

While the relative abundance of CD14⁺ cells as well as lin⁻ HLADR⁺ cells was indistinguishable between mucosal and tumor tissues, for the latter a consistent increase was observed for CD123⁺ pDCs, and a slight decrease in the abundance of cross-presenting cDC1s (FIG. 3B), as has been reported previously. When the expression of key phenotyping markers was systematically assessed across all canonical myeloid subsets, very heterogenous expression patterns were observed. All subsets except pDCs showed continuous expression of the costimulatory molecules CD40 and CD80, as well as CD86. Notably, expression of CD206 as well as PD-L1 was found across CD14⁺ monocyte/macrophage-like cells and CD1c⁺ cDC2s, as well as the CD141⁻ CD1c⁻ cell fraction (FIG. 3C).

When all 12 functional markers were compared across peripheral blood, mucosal and tumor tissues for cDC2s, cDC1s as well as CD14⁺ monocyte-like cells, tissue-infiltrating cells showed a markedly different phenotype relative to their circulating counterparts, but were relatively similar between inflamed mucosa and tumor tissues (FIG. 3D). Importantly, comparable expression patterns were observed for the commonly used M2 marker CD206 (FIG. 3E) both on CD1c⁺ cDC2s and CD14⁺ cells, challenging the notion that M2-like phenotypes are a specific hallmark of the tumor microenvironment. Also, CD163 expression was similar between the two tissue sources, suggesting similar abundance of DC3s. Similarly, comparable downregulation of CX3CR1 was observed, which regulates myeloid cell migration towards CX3CL1 (FIG. 3E).

However, while cDC2s and CD14⁺ monocyte/macrophage-like cells from both tissue sources showed increased expression of the costimulatory molecule CD80 relative to peripheral blood, the levels of CD80 were highest in tumor-infiltrating cells (FIG. 3F). Similarly, elevated co-expression of CD40 and PD-L1 was found in cDC2s derived from tumors, suggesting that an activated profile of APCs including both co-stimulatory and co-inhibitory receptors is a distinct hallmark of the tumor microenvironment.

Comprehensive Single-Cell RNAseq Analysis of SCC and Inflamed Reference Tissues Reveals Subset-Specific Cytokine Modules in the APC Compartment

While the disclosed cytometric profiling identified ICOS⁺ HLA-DR⁺ Tregs and activated CD40⁺ PD-L1⁺ cDC2s as a tumor-specific population of immune cells, this analysis overall showed that both the T cell as well as the APC compartment display remarkably congruent phenotypes between SCC tumor tissues and inflamed tissues of the same anatomic origin. Thus, at least some of the cellular phenotypes that have been previously described as being tumor-unique can also be induced by a general inflammatory environment.

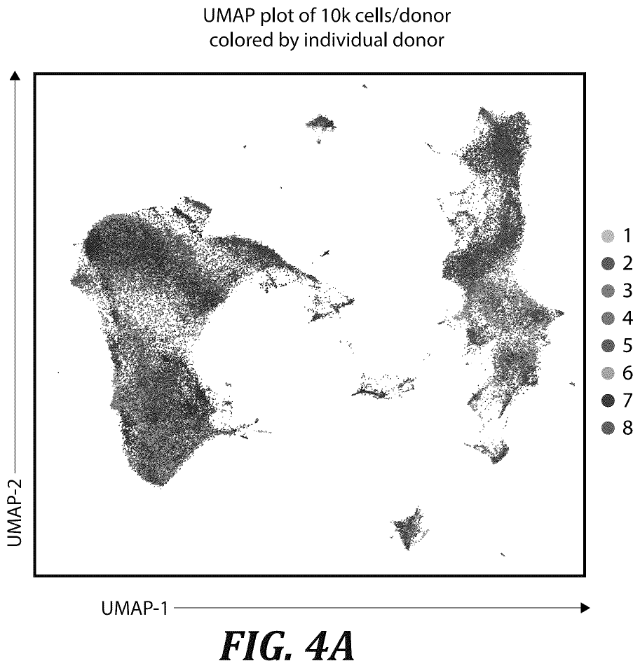

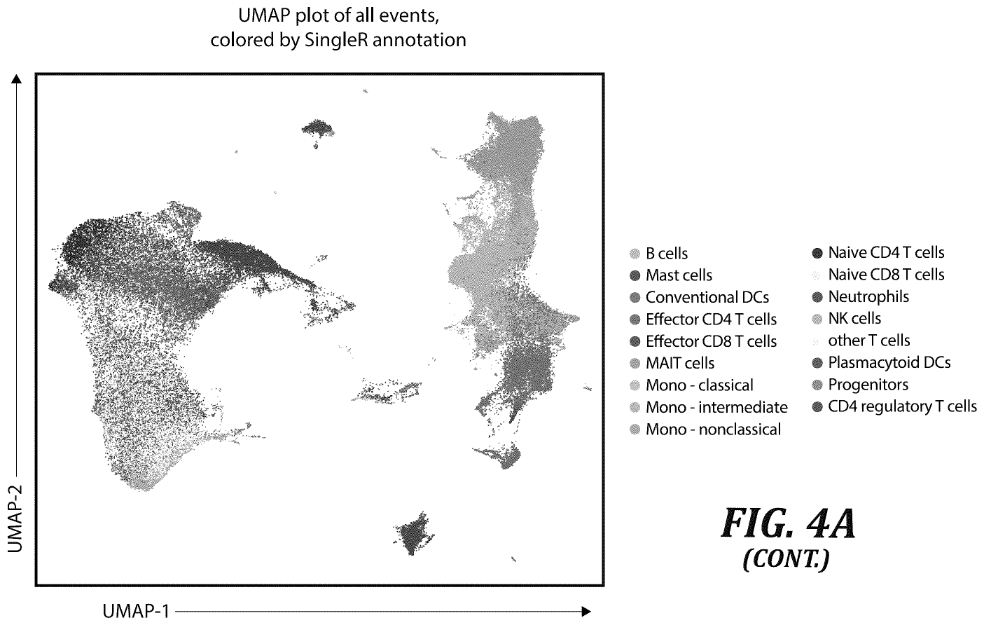

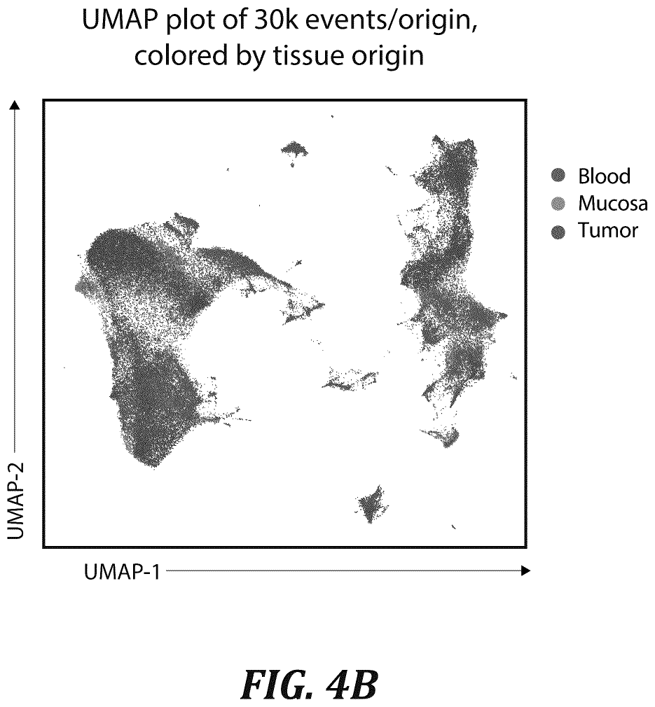

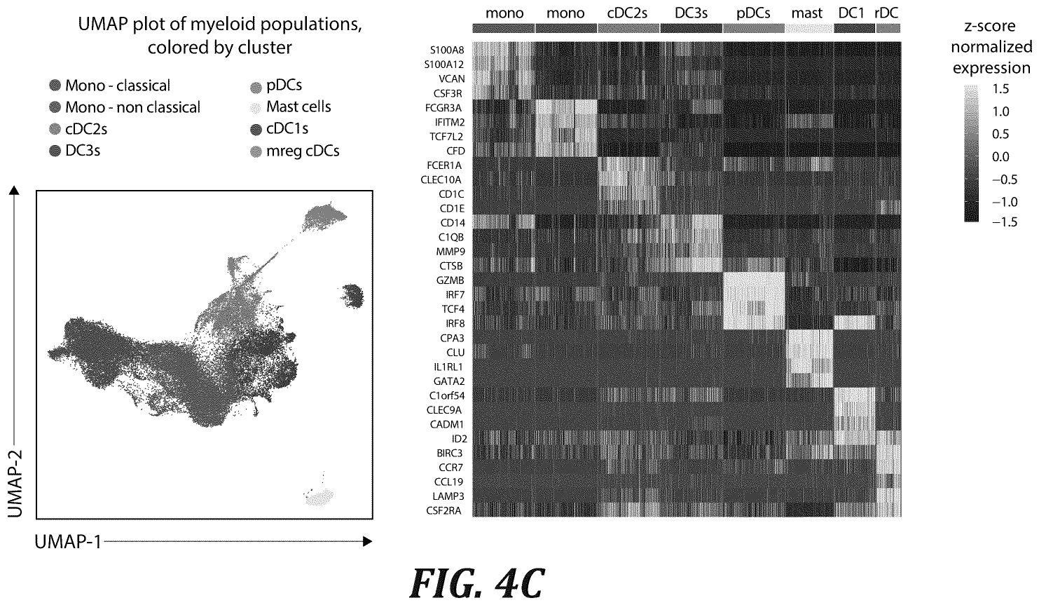

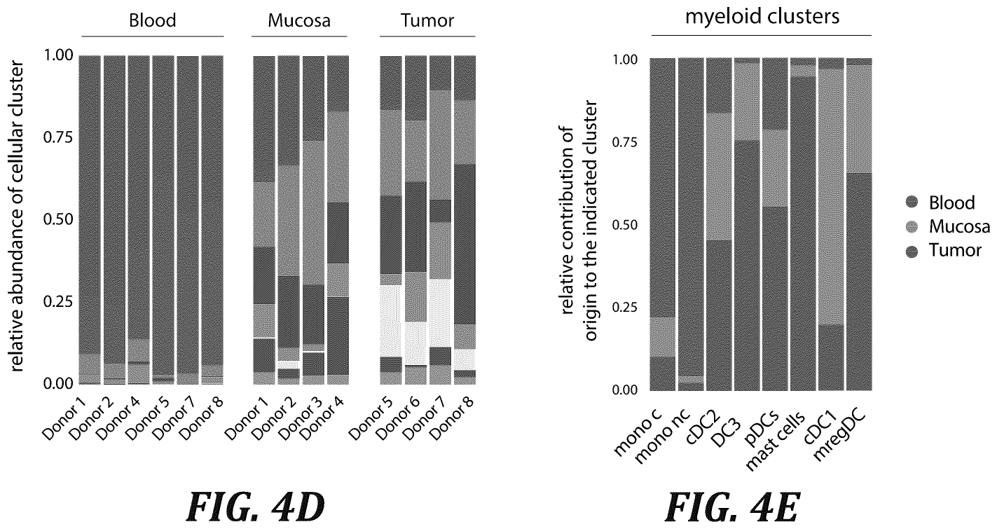

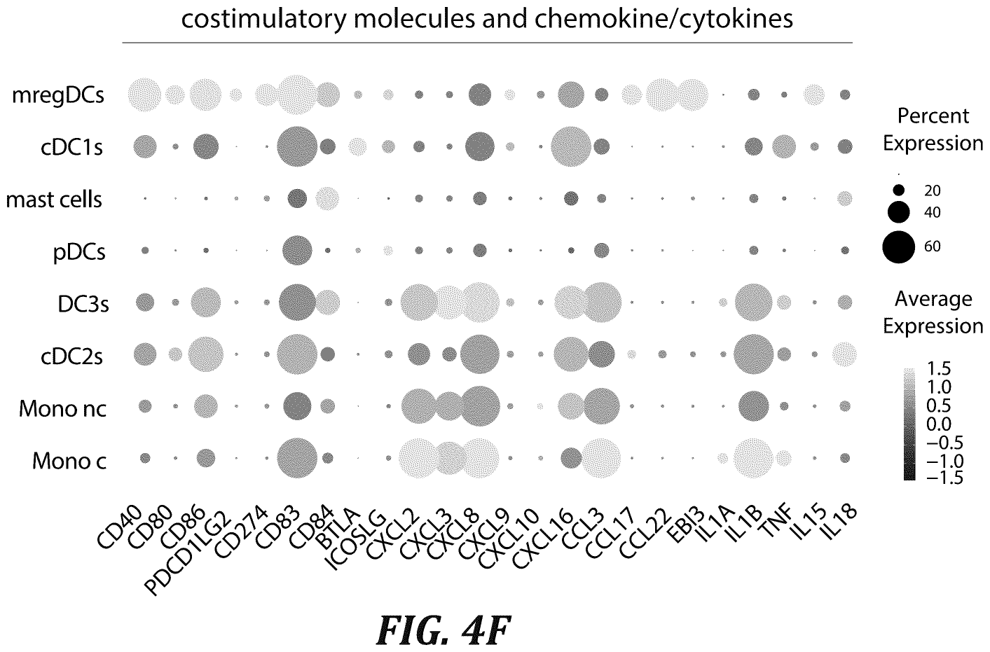

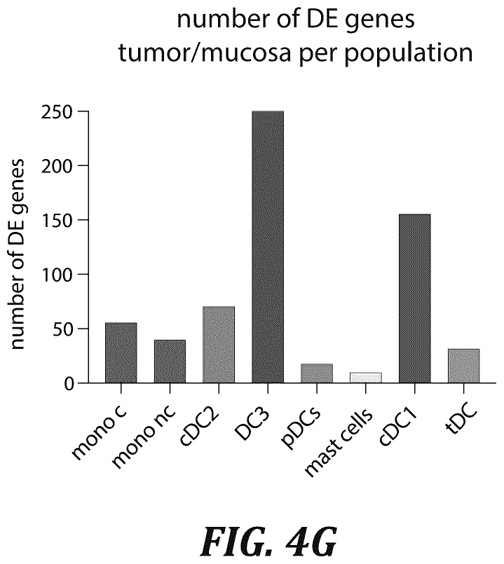

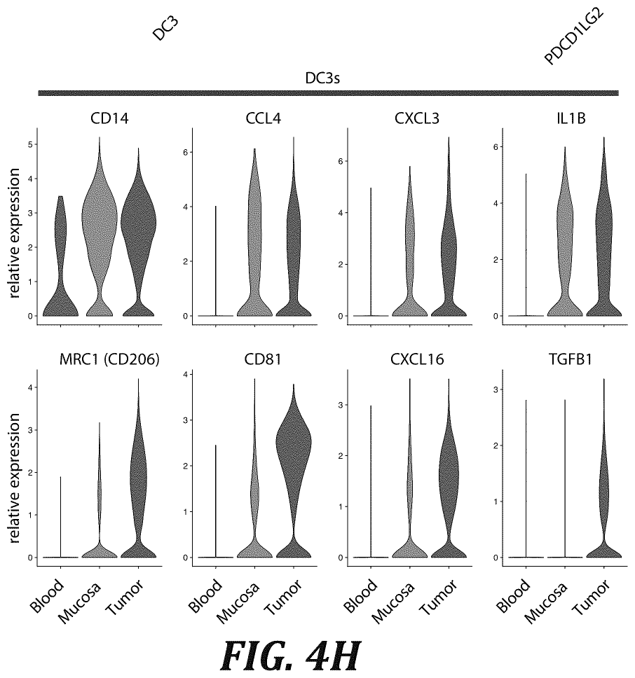

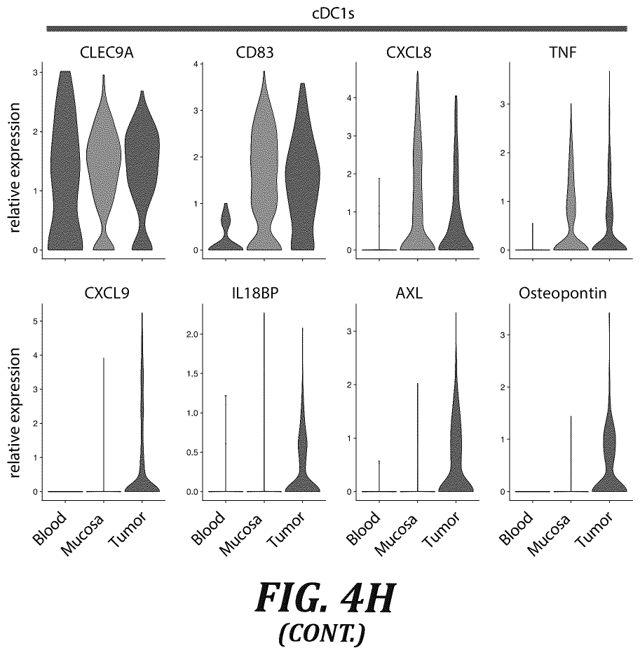

To obtain an unbiased and in-depth view of immune phenotypes that might be unique to the SCC microenvironment, single-cell RNA sequencing (scRNA-seq) analysis of sorted pan CD3⁺ T cells as well as lineage-HLADR⁺ pan APCs was performed from multiple donors of both inflamed oral tissue and OSCC samples with matched blood. After quality control and data integration using Harmony a total of approximately 150,000 cells was obtained, providing one of the most comprehensive data sets covering human tissue-derived T cells and APCs to date. For visualization uniform manifold approximation and projection (UMAP) were used. To annotate the main cellular populations in the data in an automated fashion, SingleR was used showing canonical T cell populations positioned on the left side and myeloid APC populations positioned on the right side of the UMAP plot (FIG. 4A). When the cells were grouped by tissue origin, it was observed that mucosa and tumor-derived cells co-mingled, but were mostly separate from peripheral blood on the UMAP plot (FIG. 4B). This further substantiated the conclusion from the flow cytometry data that mucosa- and tumor-infiltrating cells show a large phenotypic overlap relative to blood.

Next, an in-depth analysis was performed on the APC compartment. Subsetting and re-clustering revealed eight distinct populations, which were mapped to established APC lineages (FIG. 4C) with three notable observations: First, cells with a cDC2 phenotype were split into two populations, with one of these clusters showing an activated phenotype and intermediate expression of CD14, in line with recent reports describing DC3s as a distinct lineage of dendritic cells, which in the past often hast been called “inflammatory DCs”. Second, a previously unknown population of cells with a very discrete DC phenotype were identified, expressing high levels of CCR7, CCL19 and the GM-CSF receptor (CSF2A). Very recently, cells with this phenotype have been dubbed “mregDCs”, and this nomenclature is adopted throughout herein. However, it is important to note that these mregDCs were present both in mucosal as well as tumor tissues with comparable abundance. Third, a relatively large population of mast cells were found that express the signature gene CLU (mast cell carboxypeptidase A) as well as GATA2.

When we assessed the relative distribution of clusters across the different donors and tissue sources, we observed relatively consistent patterns (FIG. 4D). Peripheral blood samples were primarily comprised of monocytes with a classical (i.e. CD14⁺) and non-classical (i.e. FCGR3A⁺) phenotype, as well as cDC2s and pDCs (FIG. 4E). In contrast, all mucosal and tumor tissues harbored a large proportion of DC3s, cDC1s as well as mregDCs. Surprisingly, mast cells were primarily found in SCC tumor tissue (FIG. 4E).

To characterize the functional profile of these tissue-derived APCs, expression of key co-stimulatory/inhibitory genes, chemokines involved in T cell attraction, as well as cytokines involved in T cell differentiation for the mucosa- and tumor-derived cells were plotted (FIG. 4F). For both the mast cell and pDC cluster, generally low to absent expression of these genes was found, suggesting that despite their expression of HLA-DR these cell types might not play a major role in T cell recruitment and differentiation. In turn, mregDCs expressed the highest levels of costimulatory molecules, as well as CCL17/22 and EBI3 (one of the subunits of IL-27), in line with the first report describing this DC phenotype. Furthermore, it was noted that modules of lymphocyte-attracting chemokines (CXCL2/3 as well as CXCL16 and CCL3) were mostly shared among the monocyte, cDC2 and DC3 clusters, with monocyte/macrophage like cells expressing the highest levels.

Next, it was assessed how the functional properties of these APC clusters changed in the tumor microenvironment relative to the inflamed mucosa. When the number of differentially expressed (DE) genes as identified was plotted using model-based analysis of single-cell transcriptomics (MAST) it was found that the only two clusters showing a large adaptation of their transcriptomic landscape in the tumor (i.e. more than 150 genes) were DC3s and cDC1s (FIG. 4G). Of note, mregDCs, which have been postulated to be critical for anti-tumor immune responses in these analysis, showed only 20-30 transcripts with changed expression patterns, suggesting that their function does not change drastically in the tumor microenvironment relative to general tissue inflammation.

When differential gene expression was investigated in more detail, it was found that DC3s expressed CD14 across all tissue sources and showed a general tissue-specific inflammatory profile both in mucosa and tumors, with high expression of CCL4, CXCL3 and IL-1B (FIG. 4H, left panel). Among the highest tumor-enriched genes were MRC1 (CD206), the costimulatory molecule CD81, the chemokine CXCL16 and TGFB1. For cross-presenting cDC1s, the general tissue-specific inflammatory genes were CD83, CXCL8 (IL-8) as well as TNF, while the tumor-specific genes were CXCL9, IL-18BP, AXL and Osteopontin (FIG. 4H, right panel).

Overall, these comprehensive single-cell data suggest that the changed expression of a relatively small set of key immunomodulatory molecules specifically in tumor-infiltrating DC3s and cross-presenting cDC1s might be key to shaping the local anti-tumor T cell response.

NicheNet Analysis Reveals Subset-Specific Crosstalk Between Tumor-Infiltrating Myeloid APCs and T Cells

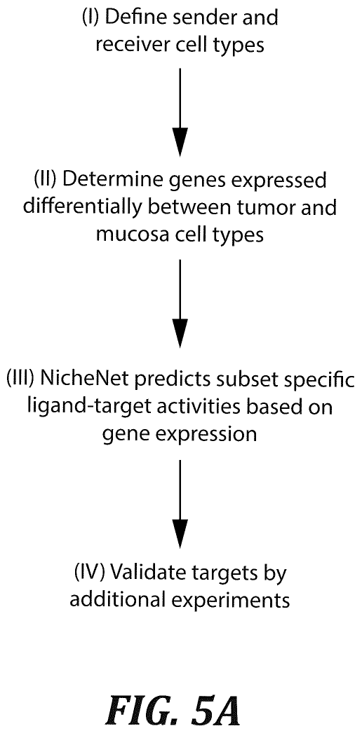

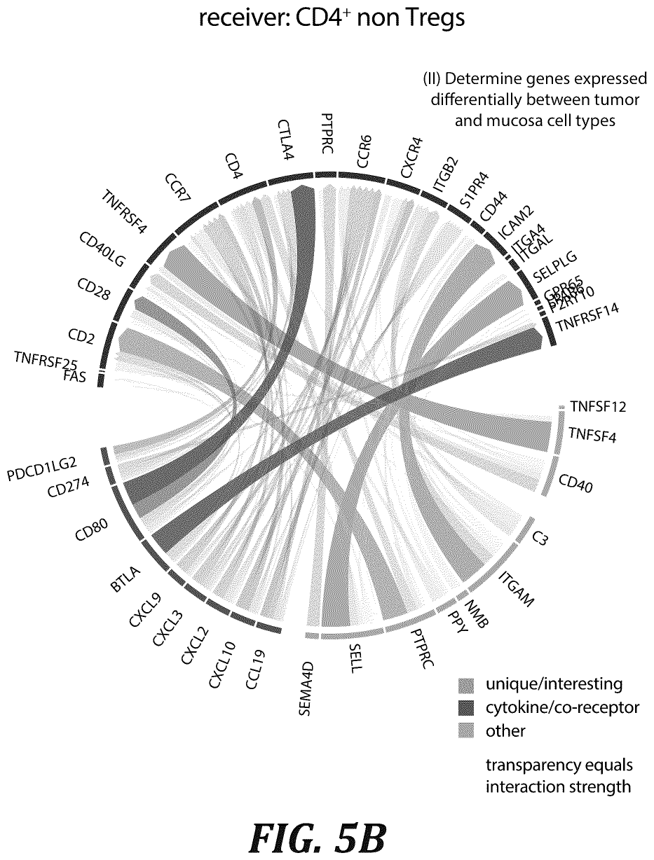

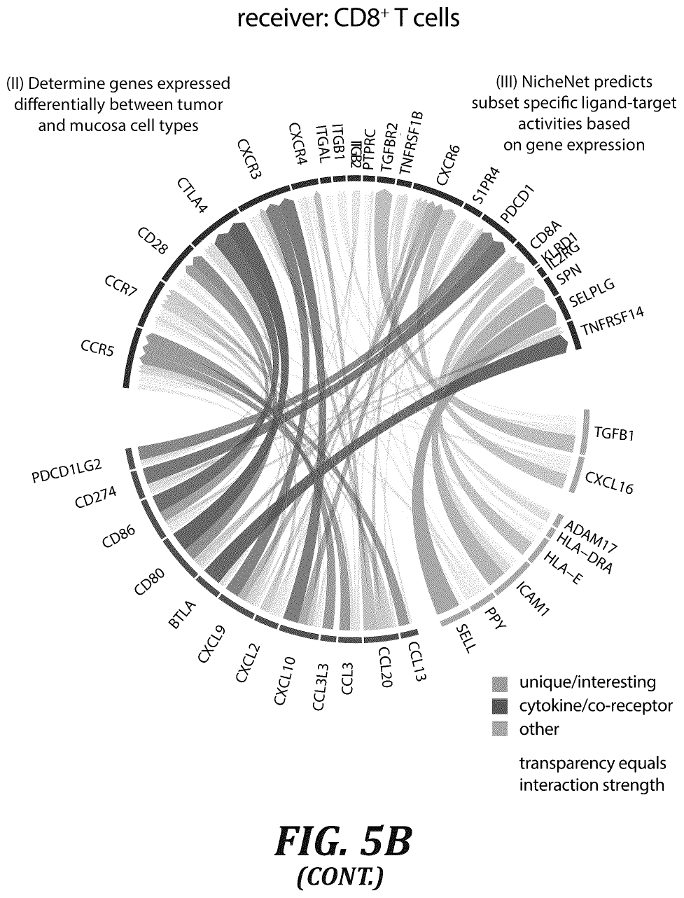

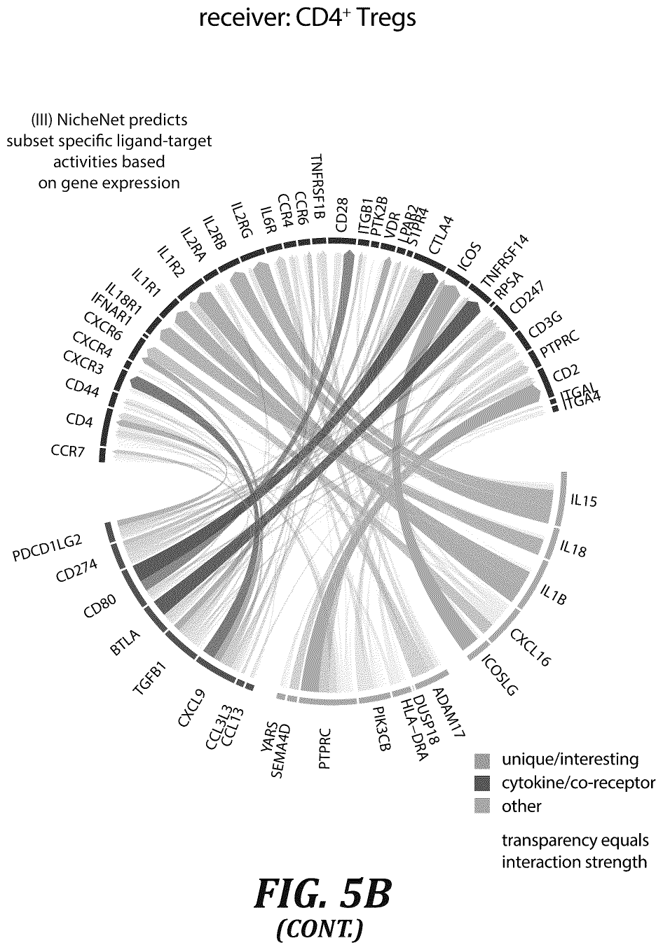

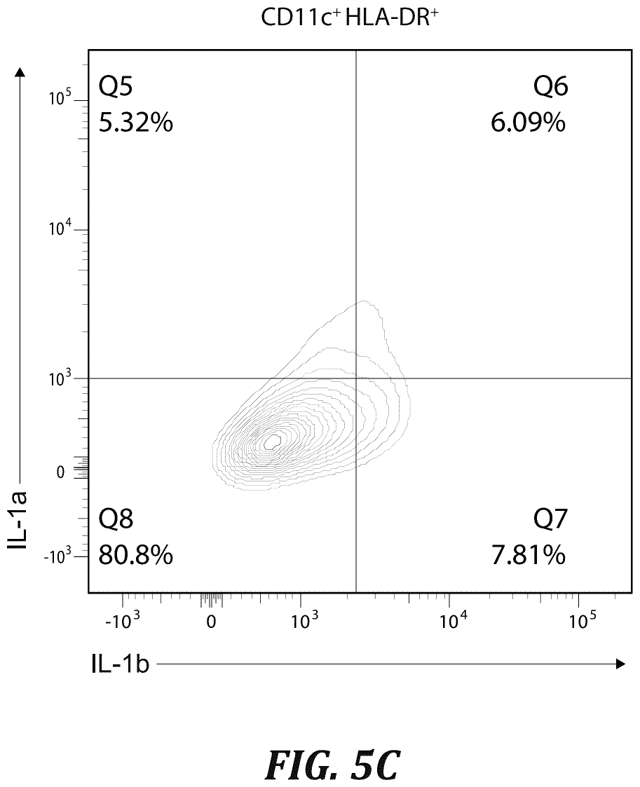

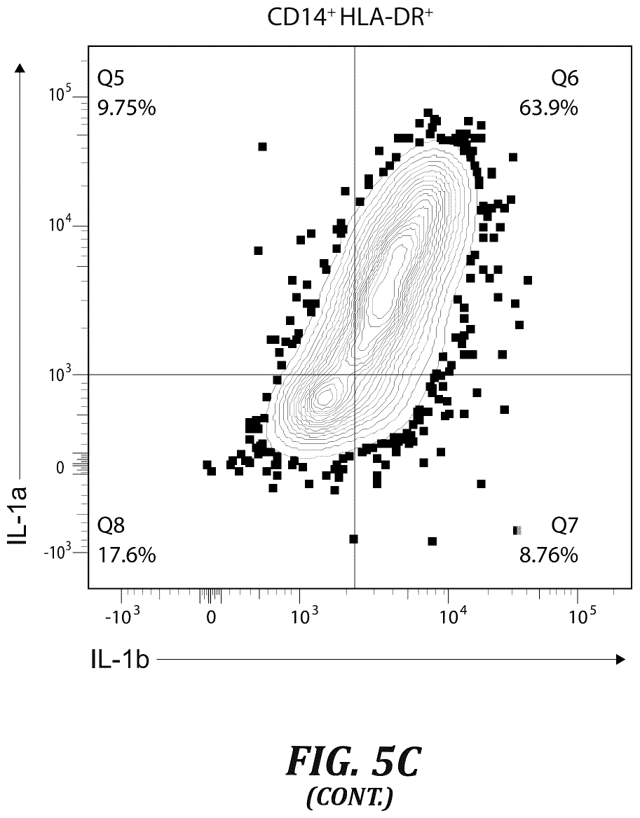

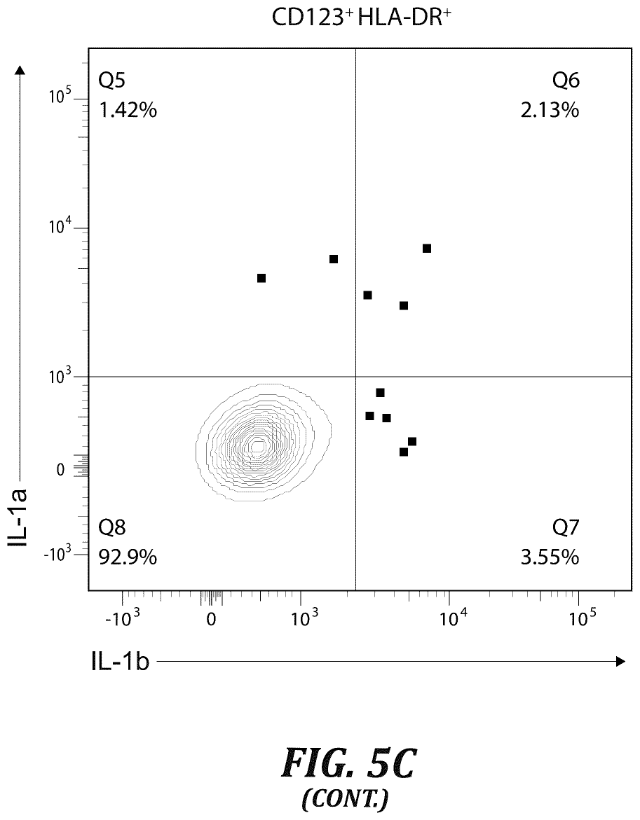

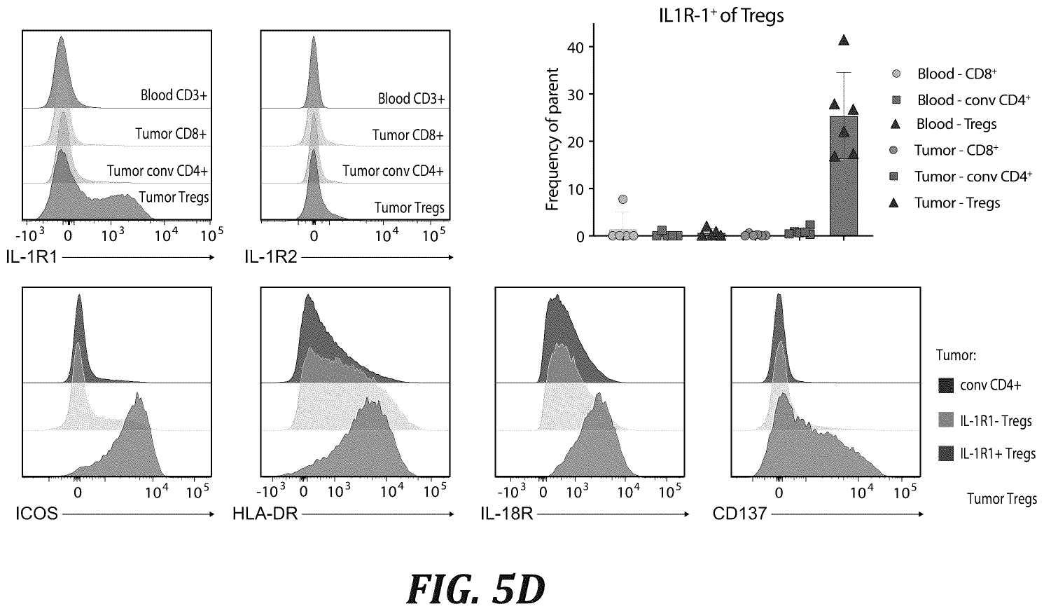

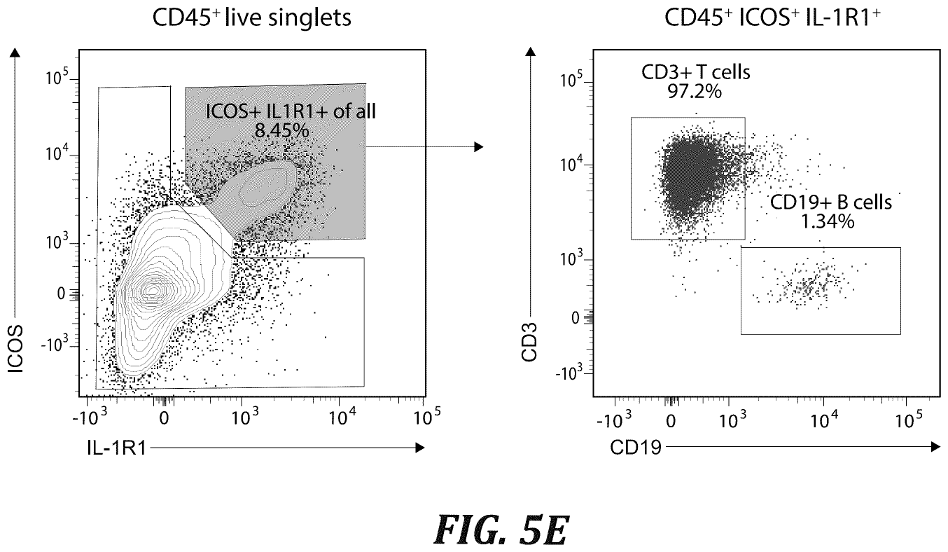

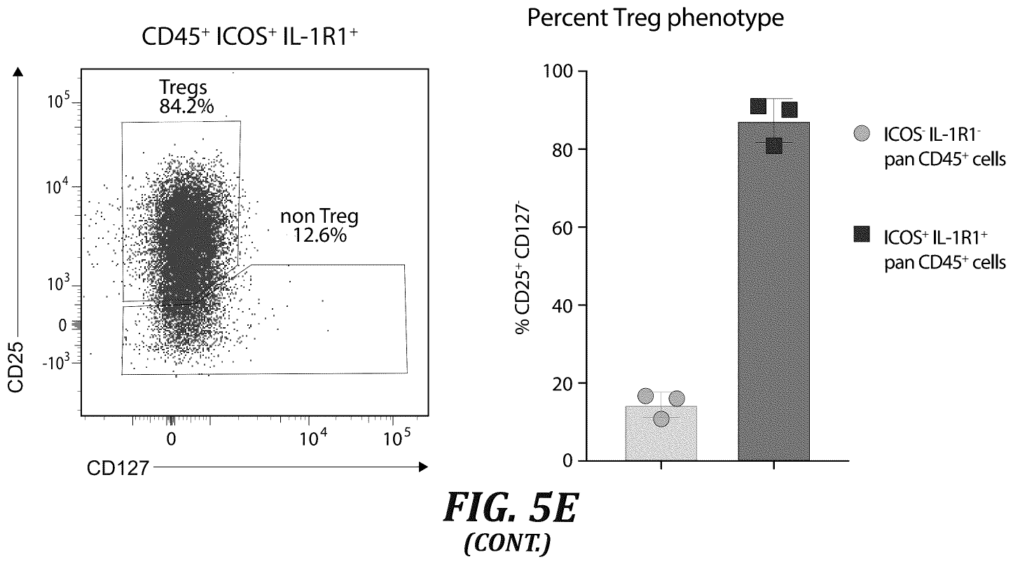

Given that this scRNA-seq data set comprises a large number of both APCs and T cells the next goal was to determine the crosstalk between these two populations in the human tumor microenvironment using NicheNet, a novel method for modeling intercellular communication by incorporation of downstream regulatory gene networks. As an input, all myeloid APC clusters were used excluding pDCs and mast cells as the sender population, and the CD4⁺ helper T cell, CD8⁺ cytotoxic T cell, and CD4⁺ Treg clusters were used as separate receiver populations. Importantly, the NicheNet workflow allows to incorporate a DE gene test for the target gene network, which was used to identify genes that were differentially expressed specifically in tumor-derived T cells relative to the inflamed mucosal tissue samples (workflow outlined in FIG. 5A).

For each T cell subset, the analysis focused on the top 20 ligand-receptor pairs identified by NicheNet and visualized these as circos plots, with the transparency of the connection reflecting the interaction strength of the respective pair (FIG. 5B). NicheNet predicted that in the tumor microenvironment all T cell lineages consistently received four costimulatory and co-inhibitory signals: PD-L1 and PD-L2 signals, CD80 signaling to CD28 and CTLA-4, and BTLA signaling to Herpesvirus entry mediator (HVEM, TNFRSF14). However, several pathways were predicted to be exclusive to a given T cell lineage: only CD4⁺ helper T cells integrated signals via the OX40L/OX40 axis, while only CD8⁺ cytotoxic T cells integrated signals from the chemokine CXCL16 and TGFB1 (FIG. 5B, orange connections). Remarkably, NicheNet suggested four ligands to be sensed by the Treg population only: ICOS ligand (ICOSLG) via ICOS, the cytokines IL-15 through the IL2RA and IL2RB receptor complex, and the pro-inflammatory cytokines IL-18 via the IL-18-R1 and IL-1B via the IL-1 receptors type 1 and type 2.

While IL-18 has been reported to have a distinct function in tissue-resident Tregs, and very recently the IL-18 binding protein (IL-18BP) has been identified as a potential immune checkpoint, the IL-1/IL-1R1 axis so far has not been implicated in Treg function in the human tumor microenvironment. Thus, the expression patterns predicted by NicheNet were validated. To confirm the presence of the two different IL-1 isoforms on protein level in tumor-derived APCs a short ex-vivo culture was performed in the presence of Brefeldin A only. A large proportion of CD14⁺ monocyte/macrophage like cells expressed IL-1β as well as IL-1α protein, as did up to 20% of cDC2s (FIG. 5C). As expected, CD123⁺ pDCs did not express IL-1α/β. When protein expression of the corresponding receptors on was measured T cells, IL-1R1 and IL-1R2 by flow cytometry, it was found that indeed both were specifically expressed by tumor-infiltrating Tregs, but neither by tumor infiltrating CD4⁺ helper T cells or CD8⁺ T cells, nor by T cells in the peripheral blood (FIG. 5D). While between 20 and 50% of the Tregs expressed IL-1R1, the expression of IL-1R2, which is thought to be a decoy receptor for IL-1 signaling was much lower. Of note, most IL-1R1⁺ Tregs were co-expressing ICOS and HLA-DR (Figure thus overlapping with the phenotype that FAUST identified as unique to the tumor microenvironment (FIG. 2A). Also, IL-1R1⁺ Tregs (compared to IL-1R1⁻ Tregs or CD4⁺ helper T cells) expressed high levels of the IL-18R1, and showed enriched expression of CD137 (4-1BB), which recently has been suggested as a pan-cancer Treg target.

When it was assessed whether the combined expression of IL-1R1 and ICOS could be used to specifically identify Tregs among all tumor-infiltrating CD45⁺ pan immune cells, it was found that 80-90% of cells in the pan CD45⁺ IL-1R1⁺ ICOS⁺ gate were CD3⁺ CD4⁺ CD25⁺ CD127 Tregs (FIG. 5E).

IL-1R1-Expressing Tregs Represent a Functionally Distinct Treg Population in the Human Tumor Microenvironment

Expression of IL-1R1 has been suggested to be a feature of activated Tregs, though there seems to be no difference in suppressive capacity between IL-1R1⁺ and IL-1R1⁻ Tregs. Based on present data, the detection efficiency of IL-1R1 transcript using a standard whole transcriptome approach (WTA) was approximately 10-fold lower than actual protein expression, which due to the modest capture sensitivity is the case for many transcripts, depending on the scRNA-seq platform used. Thus, a targeted transcriptomics experiment was performed using 495 pre-selected genes on sorted IL-1R1⁺ and IL-1R1⁻ Tregs, as well as conventional CD4 and CD8 T cells derived from three SCC tumor donors.

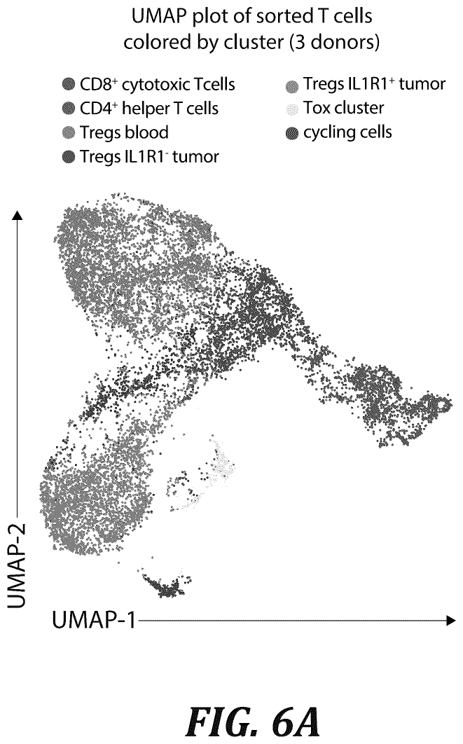

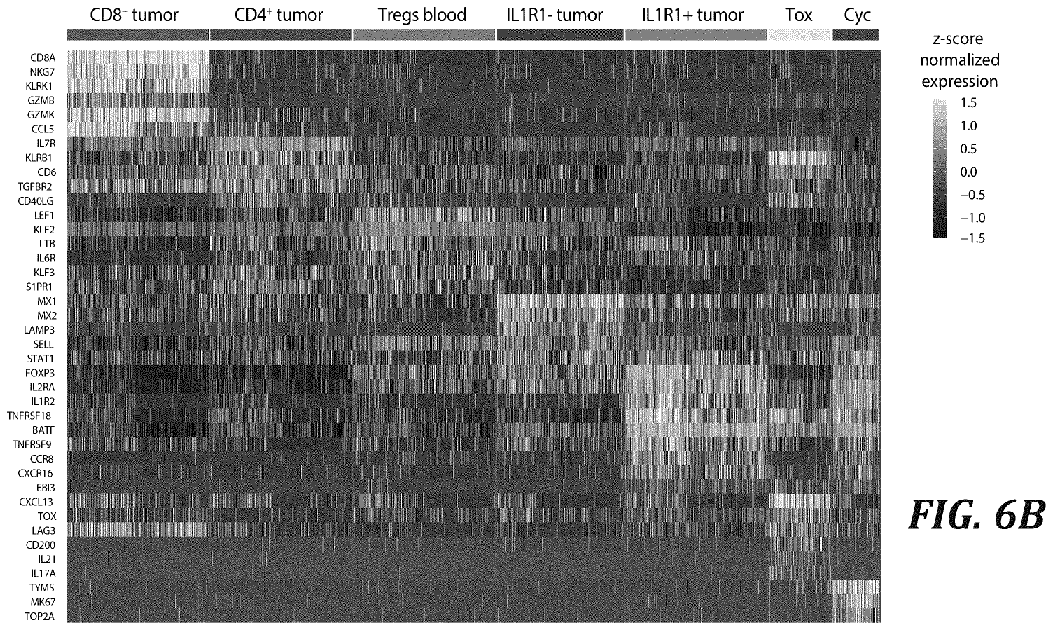

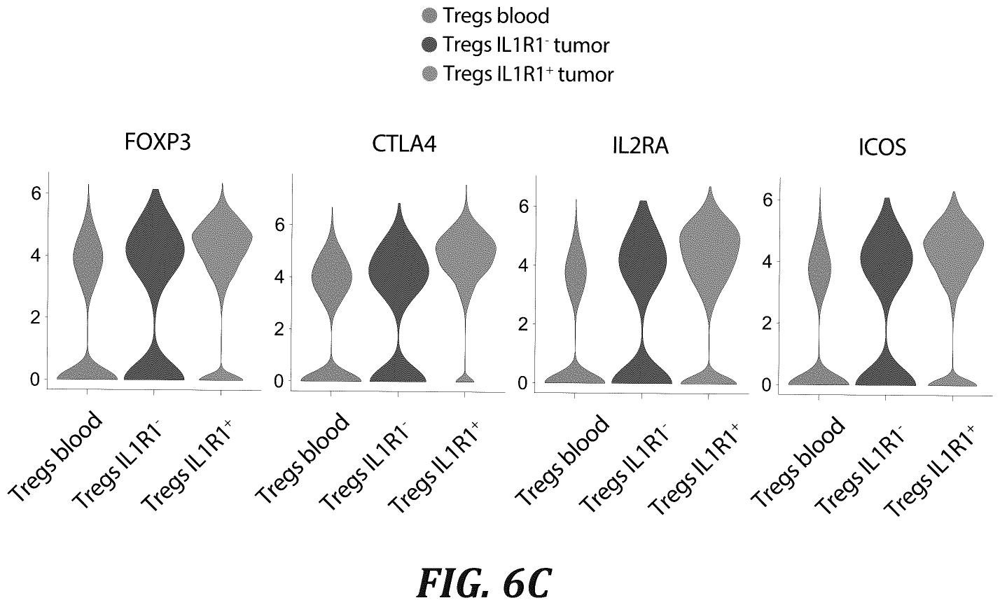

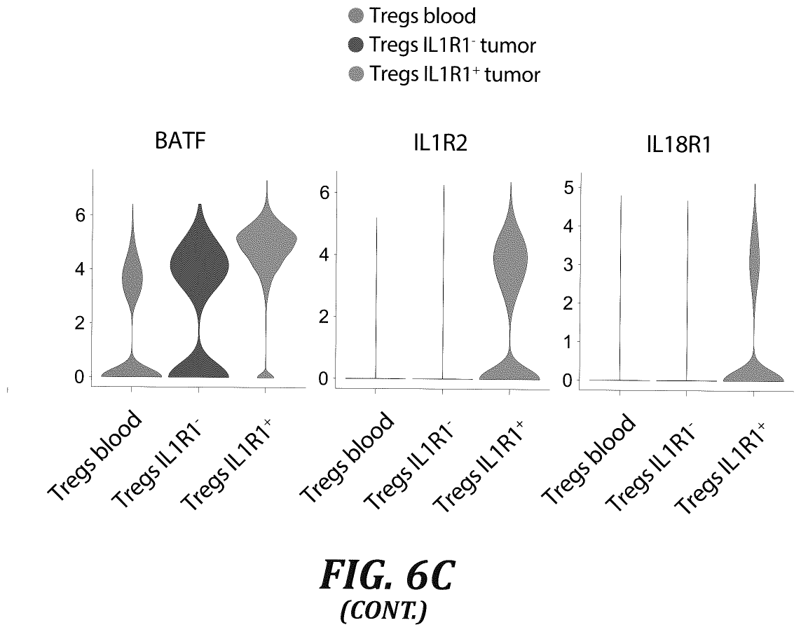

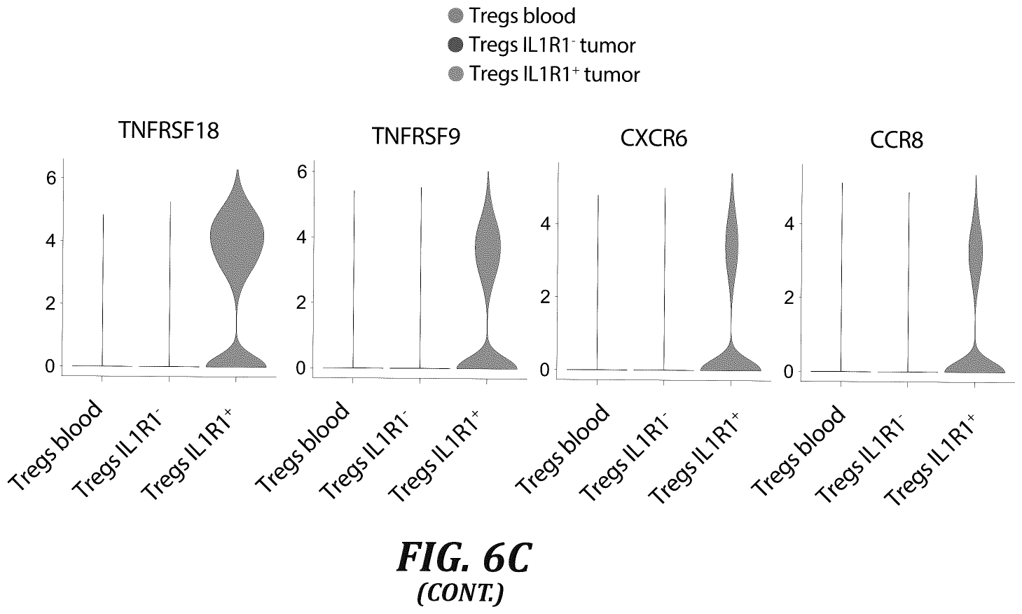

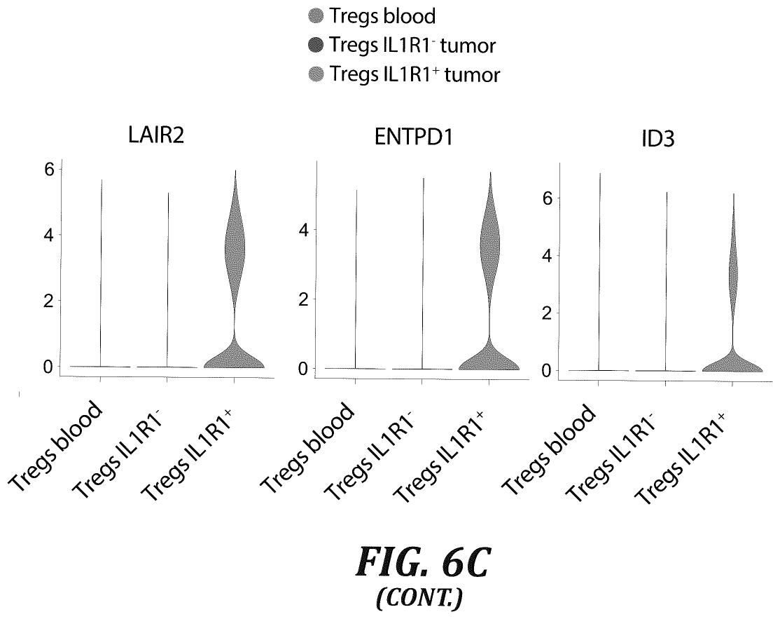

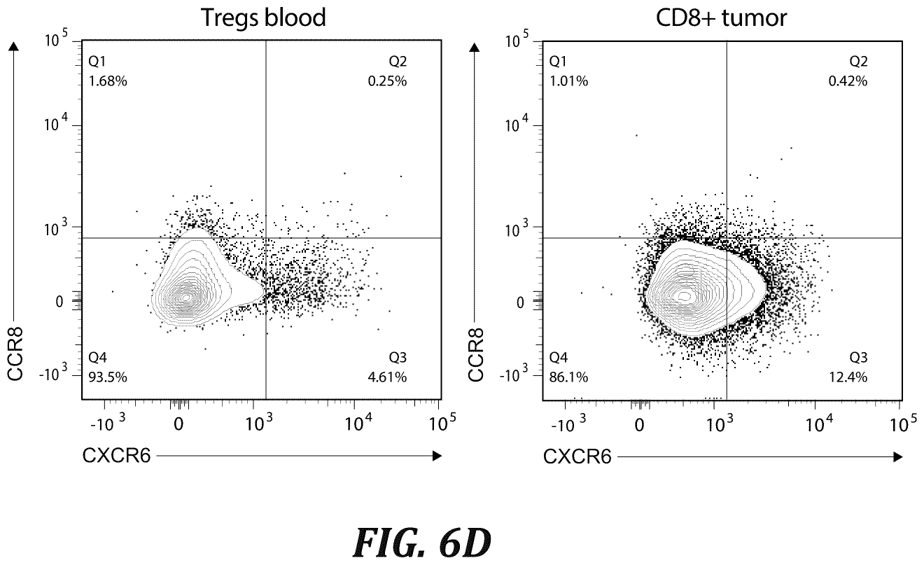

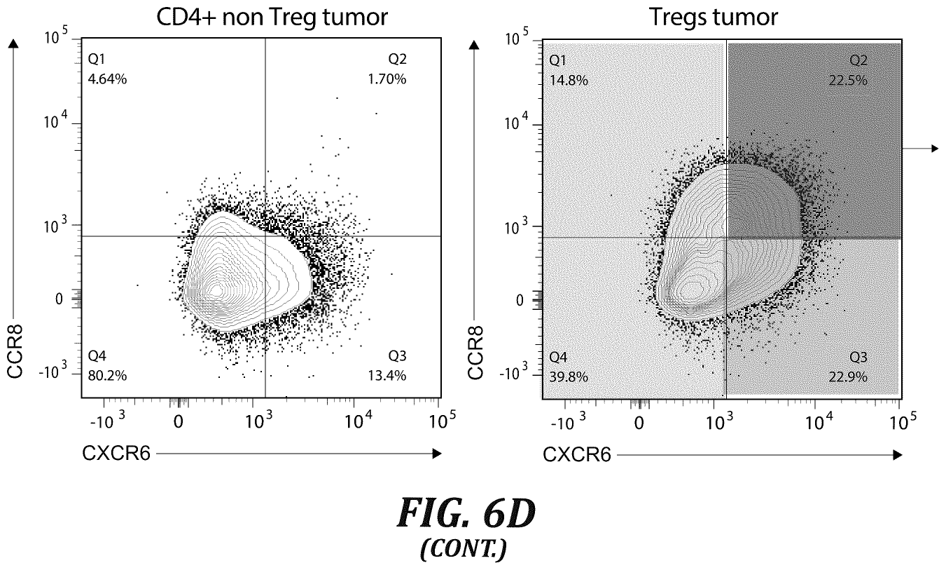

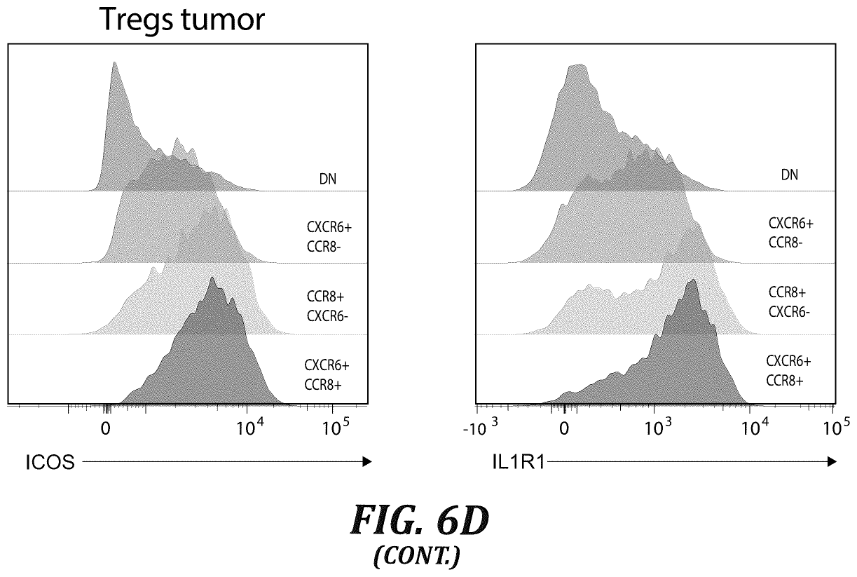

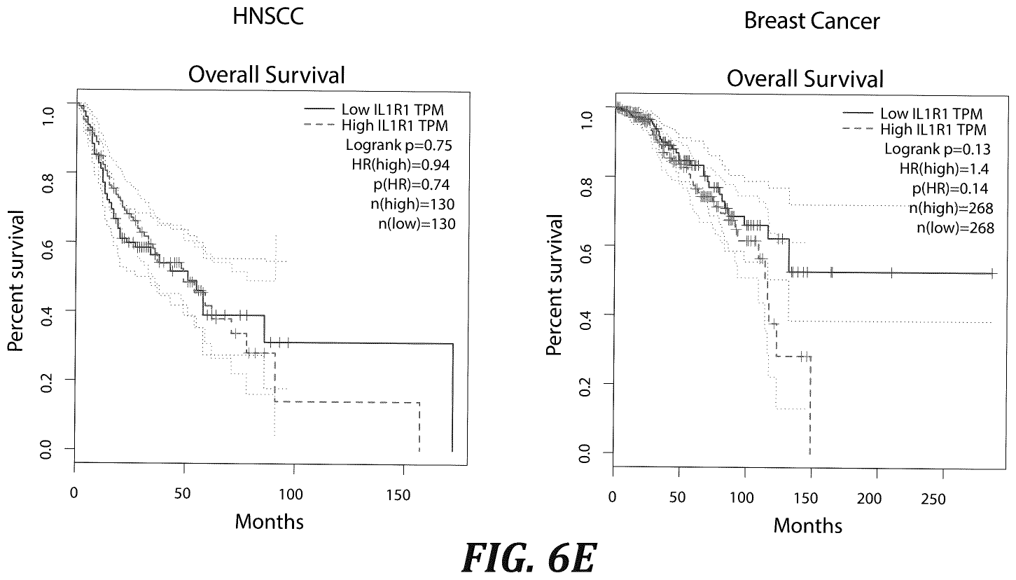

Unbiased clustering identified 7 T cell populations (FIG. 6A) with discrete gene expression profiles, including a cluster of “exhausted” T cells marked by the transcription factor TOX (FIG. 6B). Importantly, two populations of regulatory T cells were identified in the tumor that were distinct from peripheral blood Tregs. The cluster corresponding to IL-1R1+ Tregs (orange) was marked by high expression of TNFRSF18 (Glucocorticoid-induced TNF receptor, GITR), TNFRSF9 (4-1BB), the chemokine receptors CXCR6 and CCR8 as well as the transcription factor ID3, which has been implicating in the differentiation for the tissue-resident Treg program (FIG. 6C).

Having determined that ICOS⁺ IL-1R1⁺ Tregs form a distinct and targetable population the next objective was to elucidate how these cells might be recruited to the SCC microenvironment. The chemokine receptors CCR8 and CXCR6 detected by transcript in the tumor-infiltrating IL-1R1⁺ Treg cluster have been previously implicated in Treg recruitment, and when we assessed surface protein expression by flow cytometry CD25⁺ CD127⁻ Tregs showed the highest co-expression of these receptors. Importantly, CXCR6⁺CCR8⁺ Tregs were the cells also expressing the highest levels of ICOS and IL-1R1 (FIG. 6D), suggesting that the corresponding chemokines could regulate entry to the tumor microenvironment.

Finally, it was tested whether the expression of IL-1R1 could be used to stratify tumor patients for survival. Analysis of public TCGA data showed that both for HNSCC as well as breast cancer, patients with a low expression profile of IL-1R1 survived longer than these with high expression (FIG. 6E). Thus, the present data suggests that the presence of IL-1R1 expressing Tregs could be used not only as a predictive marker for survival, but also as a potential target for specific therapeutic depletion of tumor-infiltrating Tregs.

### Discussion

Due to the success of immune checkpoint blockade as a treatment for certain tumor types, the immune milieu of the human tumor microenvironment has become a main focus of cancer research and drug development. It has been suggested that the incomplete understanding of immune cell adaptation in human solid tumor tissues is a major roadblock on the path to increase the efficiency of current checkpoint blockade therapies. Thus, there is an urgent need for novel methodologies to reveal tumor-unique biomarkers or cell populations suitable for therapeutic targeting.

Described here is a unique and comprehensive single cell atlas of T cells and antigen-presenting cells (APCs) in human squamous cell carcinoma tissue relative to general tissue inflammation, revealing two novel key aspects of the immune microenvironment in human tumors: First, within the entire HLA-DR expressing APC compartment DC3s (in the past often referred to as inflammatory DCs) and cDC1s show the largest degree of tumor-driven adaptation. Second, the combined expression of ICOS, HLA-DR and IL-1R1 marks a subset of Tregs that is uniquely found in tumor relative to inflamed tissues.

While there is a significant number of reports focusing in depth on tumor-infiltrating T cells, few studies have performed parallel analysis of both T cells and myeloid cells. Early seminal work using mass cytometry in clear renal cell carcinoma concluded that there is a broad phenotypic continuum of tumor-associated macrophages (TAM) present, and that the abundance of a CD38⁺ TAM cluster correlates with the presence of PD1⁺ CD8⁺ T cells. Also, this report and others challenged the concept that CD206 or CD163 can be used as markers to define pro-tumorigenic myeloid populations. The present data support this, since both CD206 and CD163 were broadly expressed on CD14⁺ cells as well as cDC2s not only in tumor tissues, but also in inflamed reference tissue samples.

More recent work utilizing exploratory scRNA-seq approaches in breast cancer, SCC and non-small cell lung cancer (NSCLC) biopsies further highlighted that myeloid cells broadly change their function in the tumor microenvironment, and that there is extensive crosstalk between APCs and T cells that could be exploited for therapeutic intervention. In particular, Meier et al described a new dendritic cell cluster expressing high levels of immunoregulatory genes, which they termed mregDCs and suggested to be critical for the modulation of anti-tumor immunity. The same tissue DC cluster expressing high levels of CD40, PD-L1 as well as PD-L2 and CCR7, CCL17 and CSF2RA was identified, but the present data challenge the notion that this transcriptional program is specific to the tumor environment: first, the relative abundance of these cells was similar in inflamed tissue samples and tumor tissues; second, mregDCs showed only minor changes in their transcriptome between the two tissue sources, suggesting the lack of specific adaptation to the tumor microenvironment.

Contrarily, the present comprehensive single cell profiling suggests that the functional adaptation in the tumor microenvironment (based on the number of DE genes) is highest in inflammatory cDC2s and cDC1s. The latter have been shown repeatedly to be critical for anti-tumor CD8⁺ T cell responses, while the contribution of cDC2s to anti-tumor immunity has only been appreciated more recently. One seminal study showed that cDC2s not only directly induce anti-tumor CD4⁺ T cell responses, but that depletion of Tregs can improve cDC2 function and thus tumor rejection in a mouse model. The present data confirm the previously described heterogeneity in the cDC2 compartment as well as a slight reduction in cDC1 abundance, and expand these findings significantly by defining tumor-specific cytokine modules relative to a general inflammatory response. Two of these tumor-related cytokines in cDC1s were IL-18BP and Osteopontin. IL-18BP, which is a high-affinity-decoy receptor for soluble IL-18, has been recently identified as a key molecule impairing anti-tumorigenic CD8⁺ T cell responses. Similarly, Osteopontin has been suggested as a general suppressor of T cell function. In cDC2s, one of the most enriched genes was CXCL16, the ligand for CXCR6 which has been shown to regulate migration of tissue-resident memory T cells. These observations highlight that our comparison of the tumor immune profile relative to inflamed tissue samples can reveal key signaling molecules that could be targeted for therapeutic intervention.

Furthermore, the combined analysis of APCs and T cells allowed investigation of the interaction between these immune compartments. Analysis of the actively transcribed downstream gene modules using NicheNet revealed crosstalk that was shared across all T cell subsets, such as the PD-L1/PD-1 axis, a known major player in immunosuppression. However, several pathways were found that were predicted to be specifically active only in one T cell population, such as the OX40L/OX-40 axis in CD4⁺ helper T cells, which is already being exploited as a potential therapeutic target in cancer immunotherapy. Strikingly, three cytokine signals were predicted to be sensed only by Tregs, including the IL-1B/IL-1R1 axis, which is typically considered to be a pro-inflammatory signal.

The concept of specific depletion or functional modification of tumor-infiltrating Tregs as a promising anti-tumor therapy is well accepted, but systemic depletion of Tregs in humans is unfeasible due to the devastating side effects of ensuing autoimmunity. However, up until now there are few studies focusing on specific biomarkers that could be used to identify only tumor-infiltrating Tregs, with CD137 (4-1BB) being described as a potential option. The present analysis suggests that ICOS⁺ HLADR⁺ IL-1R1⁺ expressing Tregs with a tissue-resident phenotype are a truly tumor-unique population, comprising a larger pool than CD137⁺ Tregs alone. Importantly, among all CD45⁺ hematopoietic cells isolated from a tumor, ICOS and IL-1R1 were identifying with up to 90% specificity only Tregs, while none of the circulating peripheral Tregs co-expressed these markers, suggesting that the combination of ICOS and IL-1R1 could be useful biomarkers for targeting.

In summary, the present data serve as a blueprint for identifying tumor-unique immune changes, and a novel combination of two biomarkers was identified for potential targeting of tumor-infiltrating regulatory T cells.

Experimental Model and Subject Details

Primary Cells

The squamous cell carcinoma (SCC) tissue samples were obtained after informed consent from otherwise treatment-naïve patients undergoing surgical resection of their primary tumor, ensuring that the immune infiltrate was not influenced by prior therapeutic interventions such as radiotherapy. Inflamed oral tissue biopsies were obtained from individuals undergoing routine dental surgeries for a variety of inflammatory conditions such as periimplantitis, periodontitis or osseous surgery. Matched peripheral blood samples were collected from each tissue donor. All study participants signed a written informed consent before inclusion in the study, and the protocols were approved by the institutional review board (IRB) at the Fred Hutchinson Cancer Research Center (IRB #6007-972 and IRB #8335). Furthermore, cryopreserved peripheral blood mononuclear cells (PBMCs) from healthy controls (Seattle Area Control Cohort) were obtained via the HIV Vaccine Trial network (HVTN) and used for titrations, panel development and as a longitudinal technical control for all flow cytometry acquisitions.

Method Details

Isolation of Leukocytes from Solid Human Tissues and Peripheral Blood

After surgical procedures, fresh tissue samples were placed immediately into a conical tube with complete media (RPMI1640 supplemented with Penicillin, Streptomycin and 10% FBS) and kept at 4° C. Samples were processed within 1-4 hours after collection based on optimized protocols adapted from Leelatian et al. Briefly, tissue pieces were minced using a scalpel into small pieces and incubated with Collagenase II (Sigma-Aldrich, 0.6 mg/ml) and DNAse (50000 Units/ml) in RPMI1640 for 30-45 minutes depending on sample size. Subsequently, the remaining pieces were mechanically disrupted by repeated resuspension with a 30 ml syringe with a large bore tip (16×1½ blunt). The cell suspension was filtered using a 70 um cell strainer, washed in RPMI1640 and immediately used for downstream procedures.

Peripheral blood samples (3-10 ml) were collected in ACD tubes and then processed using SepMate tubes (StemCell Technologies, #85450) and Lymphoprep (Stem Cell Technologies, #07851) according to manufacturer protocols. Briefly, whole blood samples were centrifuged, plasma supernatant removed and the remaining cells resuspended in 30 ml of PBS and pipetted on top of 13.5 ml Lymphoprep in a SepMate tube. After centrifugation for 16 minutes at 1200 g, the mononuclear cell fraction in the supernatant was poured into a fresh 50 ml tube, washed with PBS and immediately used for downstream procedures.

If required, cells isolated from tissue samples or from peripheral blood were frozen using either a 90% FBS/10% DMSO mixture or Cell Culture Freezing Medium (Gibco, #12648010).

Flow Cytometry and Cell Sorting

For flow cytometric analysis good practices were followed as outlined in the guidelines for use of flow cytometry (Cossarizza et al., Guidelines for the use of flow cytometry and cell sorting in immunological studies (second edition). European journal of immunology 49, 1457-1973 (2019)). Directly following isolation, cells were incubated with Fc-blocking reagent (BioLegend Trustain FcX, #422302) and fixable UV Blue Live/Dead reagent (ThermoFisher, #L34961) in PBS (Gibco, #14190250) for 15 minutes at room temperature. After this, cells were incubated for 20 minutes at room temperature with 50 μl total volume of antibody master mix freshly prepared in Brilliant staining buffer (BD Bioscience, #563794), followed by two washes. All antibodies were titrated and used at optimal dilution, and staining procedures were performed in 96-well round-bottom plates (for cell sorting in 5 ml polystyrene tubes). For sorting cells were immediately used after staining, and for analysis, the stained cells were fixed with 4% PFA (Cytofix/Cytoperm, BD Biosciences) for 20 minutes at room temperature, washed, resuspended in FACS buffer and stored at 4° C. in the dark until acquisition. If necessary, intracellular (CD68, Granzyme B) or intranuclear staining (Foxp3, KI67) was performed following the appropriate manufacturer protocols (eBioscience Foxp3/Transcription Factor Staining Buffer Set, Thermo Fisher #00-5532-00)

Single-stained controls were prepared with every experiment using antibody capture beads diluted in FACS buffer (BD Biosciences anti-mouse, #552843 and anti-rat, #552844), or cells for Live/Dead reagent, and treated exactly the same as the samples (including fixation procedures).

All samples were acquired using a FACSymphony A5 (BD Biosciences), equipped with 30 detectors and 355 nm (65 mW), 405 nm (200 mW), 488 nm (200 mW), 532 nm (200 mW) and 628 nm (200 mW) lasers and FACSDiva acquisition software (BD Biosciences). Detector voltages were optimized using a modified voltage titration approach (Perfetto et al., Quality assurance for polychromatic flow cytometry using a suite of calibration beads. Nature protocols 7, 2067-2079 (2012)) and standardized from day to day using MFI target values and 6-peak Ultra Rainbow Beads (Spherotec, #URCP-38-2K) (Mair, F. & Tyznik, A. J. High-Dimensional Immunophenotyping with Fluorescence-Based Cytometry: A Practical Guidebook. Methods in molecular biology (Clifton, N.J.) 2032, 1-29 (2019)). After acquisition, data was exported in FCS 3.1 format and analyzed using FlowJo (version 10.6.x, BD Biosciences). Doublets were excluded by FSC-A vs FSC-H gating.

All cell sorting was performed either on a FACSAria III (BD Biosciences), equipped with 20 detectors and 405 nm, 488 nm, 532 nm and 628 nm lasers or on a FACSymphony S6 cells sorter (BD Biosciences), equipped with 50 detectors and 355 nm, 405 nm, 488 nm, 532 nm and 628 nm lasers. For all sorts involving myeloid cells, an 85 nozzle operated at 45 psi sheath pressure was used, for sorts exclusively targeting T cells, a 70 μm nozzle at 70 psi sheath pressure was used. Cells were sorted into chilled Eppendorf tubes containing 500-1000 μL of complete RPMI, washed once in PBS and immediately used for subsequent processing.

Whole Transcriptome Single-Cell Library Preparation and Sequencing

cDNA libraries were generated using the 10× Genomics Chromium Single Cell 3′ Reagent Kits v2 protocol or the v3 protocol (10× Genomics). Briefly, after sorting single cells were isolated into oil emulsion droplets with barcoded gel beads and reverse transcriptase mix using the Chromium controller (10× Genomics). cDNA was generated within these droplets, then the droplets were dissociated. cDNA was purified using DynaBeads MyOne Silane magnetic beads (ThermoFisher, #370002D). cDNA amplification was performed by PCR (10 cycles) using reagents within the Chromium Single Cell 3′ Reagent Kit v2 or v3 (10× Genomics). Amplified cDNA was purified using SPRIselect magnetic beads (Beckman Coulter). cDNA was enzymatically fragmented and size selected prior to library construction. Libraries were constructed by performing end repair, A-tailing, adaptor ligation, and PCR (12 cycles). Quality of the libraries was assessed by using Agilent 2200 TapeStation with High Sensitivity D5000 ScreenTape (Agilent). Quantity of libraries was assessed by performing digital droplet PCR (ddPCR) with Library Quantification Kit for Illumina TruSeq (BioRad, #1863040). Libraries were diluted to 2 nM and paired-end sequencing was performed on a HiSeq 2500 (Illumina) or a NovaSeq 6000 (Illumina).

Targeted Transcriptomics Single-Cell Library Preparation and Sequencing

cDNA libraries were generated as described in detail in the following protocol (Erickson, J. R. et al. AbSeq Protocol Using the Nano-Well Cartridge-Based Rhapsody Platform to Generate Protein and Transcript Expression Data on the Single-Cell Level. STAR protocols). Briefly, after sorting single cells were stained with Sample-Tag antibodies (if required), washed, pooled and counted and subsequently loaded onto a nano-well cartridge (BD Rhapsody), lysed inside the wells followed by mRNA capture on cell capture beads according to manufacturer instructions. Cell Capture Beads were retrieved and washed prior to performing reverse transcription and treatment with Exonuclease I. cDNA underwent targeted amplification using the Human Immune Response Panel primers and a custom supplemental panel (listed in Suppl Table XXX) via PCR (11 cycles). PCR products were purified, and mRNA PCR products were separated from Sample-Tag PCR products with double-sided size selection using SPRIselect magnetic beads (Beckman Coulter). mRNA and Sample Tag products were further amplified using PCR (10 cycles). PCR products were then purified using SPRIselect magnetic beads. Quality and quantity of PCR products were determined by using an Agilent 2200 TapeStation with High Sensitivity D5000 ScreenTape (Agilent) in the Fred Hutch Genomics Shared Resource laboratory. Targeted mRNA product was diluted to 2.5 ng/μL and the Sample Tag PCR products were diluted to 1 ng/μL to prepare final libraries. Final libraries were indexed using PCR (6 cycles). Index PCR products were purified using SPRIselect magnetic beads. Quality of final libraries was assessed by using Agilent 2200 TapeStation with High Sensitivity D5000 ScreenTape and quantified using a Qubit Fluorometer using the Qubit dsDNA HS Kit (ThermoFisher). Final libraries were diluted to 2 nM and multiplexed for paired-end (150 bp) sequencing on a HiSeq 2500 (Illumina) or a NovaSeq 6000 (Illumina).

Quantification and Statistical Analysis

Pre-processing for WTA and targeted transcriptomics data Raw base call (BCL) files were demultiplexed to generate Fastq files using the cellranger mkfastq pipeline within Cell Ranger (10× Genomics). Whole transcriptome Fastq files were processed using the standard cellranger pipeline (10×genomics) within Cell Ranger 2.1.1 or Cell Ranger 3.x.x. Briefly, cellranger count performs read alignment, filtering, barcode and UMI counting, and determination of putative cells. The final output of cellranger (the molecule per cell count matrix) was then analyzed in R using the package Seurat (version 2.3 and 3.0) as described below. For targeted transcriptomics data, Fastq files were processed via the standard Rhapsody analysis pipeline (BD Biosciences) on Seven Bridges (sevenbridges.com). Briefly, after read filtering, reads are aligned to a reference genome and annotated, barcodes and UMIs are counted, followed by determining putative cells. The final output (molecule per cell count matrix) was also analyzed in R using Seurat (version 3.0) as described below.

Seurat Workflow for Targeted and WTA Data

The R package Seurat (Butler et al., Integrating single-cell transcriptomic data across different conditions, technologies, and species. Nature biotechnology 36, 411-420 (2018)) was utilized for all downstream analysis, with custom scripts based on the following general guidelines for analysis of scRNA-seq data (Amezquita, R. A. et al. Orchestrating single-cell analysis with Bioconductor. Nature Methods 12, 1-9 (2019)).

Briefly, for whole transcriptome data, only cells that had at least 200 genes (v2 kits) or 800 genes (v3 kits), and depending on sample distribution less than 7-15% mitochondrial genes were included in analysis. All samples were merged into a single Seurat object, followed by a natural log normalization using a scale factor of 10000, determination of variable genes using the vst method, and a z-score scaling. Principal component analysis (PCA) was used to generate 75 PCs, followed by data integration using Harmony (Kornuntsky et al). The dimensionality reduction generated by Harmony was used to calculate UMAP, and graph-based clustering with a resolution tuned to the respective data set.

For targeted transcriptomics data, separate cartridges from the same experiment were merged (if applicable), and only cells that had at least 30 genes were included in downstream analysis. After generating a Seurat object, a natural log normalization using a scale factor of 10000 was done, followed by determination of variable genes using the vst method, and a z-score scaling. PCA was used to generate 75 PCs, 30 of which were used for subsequent UMAP calculation and graph-based clustering with tuned resolution.

For all differential gene expression analysis the Seurat implementation of MAST (model-based analysis of single-cell transcriptomes) was used with the number of UMIs included as a covariate (proxy for cellular detection rate (CDR)) in the model (Finak et al., MAST: a flexible statistical framework for assessing transcriptional changes and characterizing heterogeneity in single-cell RNA sequencing data. Genome biology 16, 278, (2015)).

Data and Code Availability

The sequencing data discussed in this publication will be deposited in the NCBI's Omnibus database, and the main scripts used for data processing are available on github (github.com/MairFlo)

### Example 2

As described in Example 1, a unique expression profile was identified that uniquely distinguished Treg cells infiltrating tumor environments from circulating Tregs (e.g., Tregs in inflamed, non-cancerous tissue). The initial work was established in SSC tumors for proof of concept. To determine if this unique expression profile was applicable to Tregs infiltrating other tumor-types, primary human breast cancer tissue was investigated.

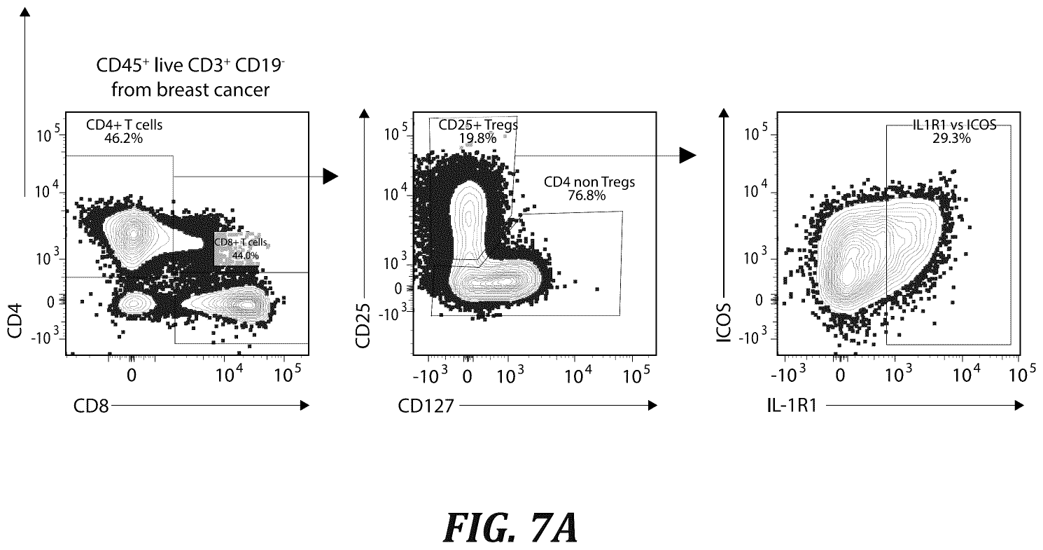

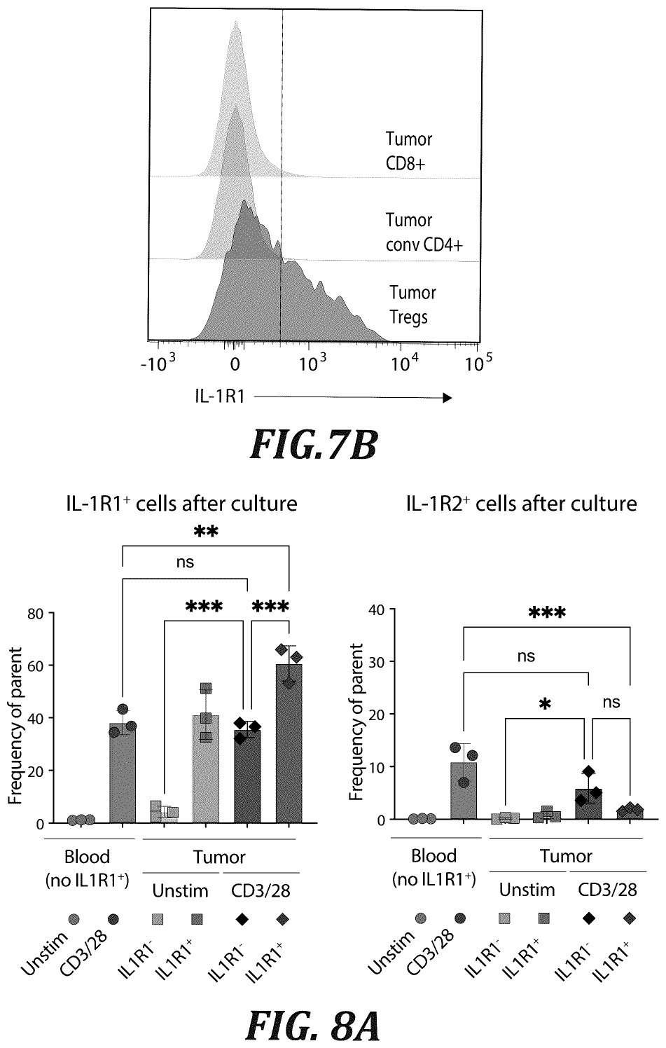

FIGS. 7A and 7B illustrate a follow up analysis of immune infiltrate in solid breast tumor tissue for expression of ICOS and IL-1R1, demonstrating that the unique expression profile is consistent in other solid tumor types. Tumor-infiltrating leukocytes were isolated from a human breast cancer tissue as described above for SCC tumor samples. FIG. 7A illustrates plots depicting gating for CD4⁺ and CD8⁺ T cells, and CD25⁺ CD127⁻ regulatory T cells (Tregs), followed by the expression pattern of ICOS and IL-1R1 on Tregs. FIG. 7B illustrates histogram plots, which show absence of IL-1R1 expression on tumor-infiltrating CD8⁺ T cells and CD4⁺ non Tregs, and expression of IL-1R1 on approximately 30% of Tregs (cut-off indicated by dashed line).

Accordingly, it is demonstrated that the observed unique expression of ICOS and IL-1R1 by tumor-infiltrating Tregs is not limited to SSC tumors but occurs in Tregs infiltrating other solid tumors, such as breast cancer tumors.

### Example 3

As described in Examples 1 and 2, a unique expression profile was identified that uniquely distinguished Treg cells infiltrating tumor environments, including from SSC and breast cancer tumors, from circulating Tregs (e.g., Tregs in inflamed, non-cancerous tissue). Additional investigation was performed to characterize these tumor-infiltrating Treg cells.

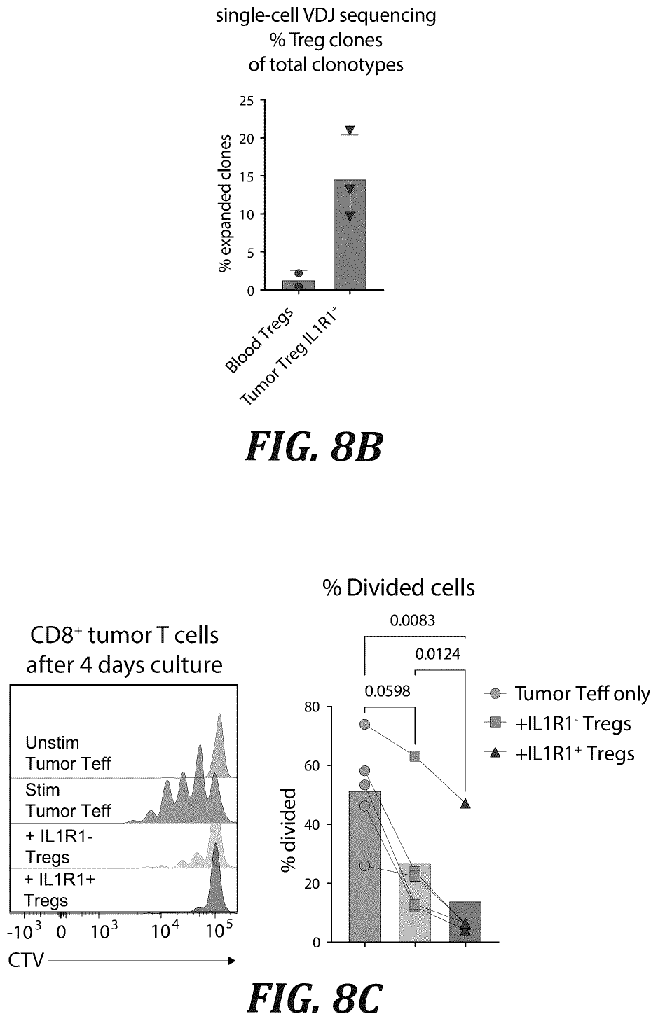

Intratumoral IL-1R1-expressing Tregs were isolated from SSC tumors and characterized in depth, demonstrating that these Tregs represent a clonally expanded Treg population with hallmarks of recent TCR activation and superior suppressive capacity. FIG. 8A graphically represents IL-1R1 expression on sorted Tregs from peripheral blood of healthy donors (“no IL-1R1+”) and HNSCC tumor tissue (“Tumor”) cultured unstimulated or in the presence of anti-CD3/28 beads for 2 days. TCR stimulation was sufficient to induce IL-1R1 expression. FIG. 8B graphically illustrates analysis of TCR diversity by single-cell VDJ sequencing within sorted IL-1R1⁺ Tregs from HNSCC tumors relative to total Tregs from matched peripheral blood. Every TCR sequence that was present in two or more cells was considered an expanded clone. FIG. 8C, left panel, shows Cell Trace Violet (CTV) dilution of sorted CD8⁺ T effector cells (Teff) derived from HNSCC tumor tissue after 4 days of culture without stimulation, with stimulation beads alone, or with an equal number of sorted tumor-derived IL-1R1⁻ and IL-1R1⁺ Tregs. The plot on the right depicts the percentage of divided T effector cells in each condition. All summary graphs are represented as mean±SD (total n=3 for stimulation assay and VDJ-sequencing, n=5 from multiple experiments for suppression assay). Statistical analyses were performed using one-way ANOVA with Tukey's multiple comparisons test.

These results demonstrate that the IL-1R1⁺ ICOS⁺ Tregs isolated from the tumor tissue constitute a clonally expansive population of super-suppressor Tregs because they inhibit T cell responses more efficiently than conventional Tregs. This further highlights the clinical significance of IL-1R1⁺ ICOS⁺ Tregs.

While illustrative embodiments have been illustrated and described, it will be appreciated that various changes can be made therein without departing from the spirit and scope of the invention.

The embodiments of the invention in which an exclusive property or privilege is claimed are defined as follows:

1. A method of specifically inhibiting or depleting solid tumor-infiltrating regulatory T cells (Tregs), comprising contacting the solid tumor with one or more agents that specifically bind inducible T cell costimulator (ICOS) and Interleukin-1 receptor type 1 (IL-1R1).

2. The method of claim 1, wherein the one or more agents comprises a bi-specific affinity reagent with a first domain that specifically binds ICOS and a second domain that specifically binds IL-1R1.

3. The method of claim 1, wherein the one or more agents comprises a first affinity reagent that specifically binds ICOS and a second affinity reagent that specifically binds IL-1R1.

4. The method of one of claims 1-3, wherein the one or more agents induces Treg cell death.

5. The method of one of claims 1-4, wherein the one or more agents is conjugated to a payload that is toxic to the tumor-infiltrating Tregs.

6. The method of claim 1, wherein the one or more agents comprise an engineered immune cell that co-expresses a first chimeric antigen receptor (CAR) that specifically binds ICOS and a second chimeric antigen receptor (CAR) that specifically binds IL-1R1, wherein the engineered immune cell requires binding by the first CAR and second CAR to activate.

7. The method of claim 6, wherein the engineered immune cell is a logic-gated CAR T cell that requires binding of the first CAR and the second CAR to induce a T cell response by the CAR T cell.

8. The method of claim 1, wherein the one or more agents comprise a logic-gated CAR T cell that co-expresses a first chimeric antigen receptor (CAR) that specifically binds one of ICOS and IL-1R1, and a second chimeric antigen receptor (CAR) that specifically binds to the other of ICOS and IL-1R1 via a bi-functional switch molecule, wherein the CAR T cell requires binding by the first CAR and second CAR to induce a T cell response by the CAR T cell.

9. The method of claim 8, further comprising contacting the solid tumor with an effective amount of the bi-functional switch molecule, wherein the bi-functional switch molecule comprises a first domain that specifically binds to the other of ICOS and IL-1R1 and a second domain that is specifically bound by the second CAR.

10. The method of one of claims 1-9, wherein inhibiting or depleting the Tregs in the solid tumor reduces immunosuppressive conditions in the solid tumor.

11. A method of treating a subject with a solid tumor, comprising administering to the subject a therapeutic composition comprising one or more agents that specifically bind inducible T cell costimulator (ICOS) and Interleukin-1 receptor type 1 (IL-1R1).

12. The method of claim 11, wherein the one or more agents comprises a bi-specific affinity reagent with a first domain that specifically binds ICOS and a second domain that specifically binds IL-1R1.

13. The method of claim 11, wherein the one or more agents comprises a first affinity reagent that specifically binds ICOS and a second affinity reagent that specifically binds IL-1R1.

14. The method of one of claims 11-13, wherein the one or more agents bind to solid tumor-infiltrating regulatory T cells Tregs and cause cell death of the Tregs in the solid tumor.

15. The method of one of claims 11-14, wherein the one or more agents is conjugated to a payload that is toxic to the tumor-infiltrating Tregs.

16. The method of claim 11, wherein the one or more agents comprise an engineered immune cell that co-expresses a first chimeric antigen receptor (CAR) that specifically binds ICOS and a second chimeric antigen receptor (CAR) that specifically binds IL-1R1, wherein the engineered immune cell requires binding by the first CAR and second CAR to activate.

17. The method of claim 16, wherein the engineered immune cell is a logic-gated CAR T cell that requires binding of the first CAR and the second CAR to induce a T cell response by the CAR T cell.

18. The method of claim 11, wherein the one or more agents comprise a logic-gated CAR T cell that co-expresses a first chimeric antigen receptor (CAR) that specifically binds one of ICOS and IL-1R1, and a second chimeric antigen receptor (CAR) that specifically binds to the other of ICOS and IL-1R1 via a bi-functional switch molecule, wherein the CAR T cell requires binding by the first CAR and second CAR to induce a T cell response by the CAR T cell.

19. The method of claim 18, further comprising contacting the solid tumor with an effective amount of the bi-functional switch molecule, wherein the bi-functional switch molecule comprises a first domain that specifically binds to the other of ICOS and IL-1R1 and a second domain that is specifically bound by the second CAR.

20. The method of one of claims 11-19, further comprising administering to the subject an additional cancer therapy.

21. The method of claim 20, wherein the additional cancer therapy comprises administration of a checkpoint inhibitor compound, an adoptive cell therapy, an anti-cancer antigen antibody or therapeutic composition.

22. The method of claim 21, wherein the checkpoint inhibitor inhibits PD-1, PD-L1, CTLA-4, LAG-3, Tim-3, or TIGIT.

23. The method of claim 22, wherein:
the immune checkpoint inhibits PD-1 and is selected from Pembrolizumab (Keytruda), Nivolumab (Opdivo), Cemiplimab (Libtayo), and the like;
the immune checkpoint inhibits PD-L1 and is selected from Atezolizumab (Tecentriq), Avelumab (Bavencio), Durvalumab (Imfinzi), and the like; or
the immune checkpoint inhibits CTLA-4 and is selected from Ipilimumab (Yervoy), and the like.

24. The method of claim 21, wherein the adoptive cell therapy comprises immune cells that improve immune response against the tumor.

25. The method of claim 24, wherein the immune cells comprise T cells or NK cells that are genetically modified to express a chimeric antigen receptor (CAR) that specifically binds a tumor associate antigen.

26. The method of claim 21, wherein the anti-cancer antigen antibody or therapeutic composition is selected from aldesleukin, altretamine, amifostine, asparaginase, bleomycin, capecitabine, carboplatin, carmustine, cladribine, cisapride, cisplatin, cyclophosphamide, cytarabine, dacarbazine (DTIC), dactinomycin, docetaxel, doxorubicin, dronabinol, duocarmycin, etoposide, filgrastim, fludarabine, fluorouracil, gemcitabine, granisetron, hydroxyurea, idarubicin, ifosfamide, interferon alpha, irinotecan, lansoprazole, levamisole, leucovorin, megestrol, mesna, methotrexate, metoclopramide, mitomycin, mitotane, mitoxantrone, omeprazole, ondansetron, paclitaxel (Taxol™), pilocarpine, prochloroperazine, rituximab, saproin, tamoxifen, taxol, topotecan hydrochloride, trastuzumab, vinblastine, vincristine, vinorelbine tartrate, and the like.

27. The method of one of claims 11-26, wherein the solid tumor is a squamous cell carcinoma (SCC) or a breast cancer tumor.

28. A composition comprising an engineered immune cell that co-expresses a first chimeric antigen receptor (CAR) that specifically binds ICOS and a second chimeric antigen receptor (CAR) that specifically binds IL-1R1, wherein the engineered immune cell requires binding by the first receptor and second receptor to activate.

29. The composition of claim 28, wherein the engineered immune cell is a logic-gated CAR T cell that requires binding of the first CAR and the second CAR to induce a T cell response by the CAR T cell.

30. The composition of claim 28 or claim 29, wherein the composition is formulated for systemic administration.

31. A composition comprising a logic-gated CAR T cell that co-expresses a first chimeric antigen receptor (CAR) that specifically binds one of ICOS and IL-1R1, and a second chimeric antigen receptor (CAR) that specifically binds to the other of ICOS and IL-1R1 via a bi-functional switch molecule, wherein the CAR T cell requires binding by the first CAR and second CAR to induce a T cell response by the CAR T cell.

32. The composition of claim 31, wherein the bi-functional switch molecule comprises a first domain that specifically binds to the other of ICOS and IL-1R1 and a second domain that is specifically bound by the second CAR, and the CAR T cell requires simultaneous binding by the first domain to the other of ICOS and IL-1R1 and the second domain to the second CAR to induce a T cell response by the CAR T cell.

33. A method of detecting the presence of tumor-infiltrating Treg cells in a tumor environment, comprising:
contacting a sample comprising tumor cells obtained from a subject with a solid tumor with one or more agents that specifically bind inducible T cell costimulator (ICOS) and Interleukin-1 receptor type 1 (IL-1R1), wherein the one or more agents are detectably labeled; and
detecting binding of the one or more agents to a cell in the sample, wherein binding the one or more agents to a cell in the sample indicates the presence of tumor-infiltrating Treg cells in the tumor environment in the subject.

34. The method of claim 33, wherein the one or more agents are agents comprises a bi-specific affinity reagent with a first domain that specifically binds ICOS and a second domain that specifically binds IL-1R1.

35. The method of claim 33, wherein the one or more agents comprises a first affinity reagent that specifically binds ICOS and a second affinity reagent that specifically binds IL-1R1.

36. The method of claim 35, wherein the first affinity reagent produces a first detectable signal and the second affinity reagent produces a second affinity signal that is different from the first detectable signal.

37. The method of one of claims 33-36, wherein the detecting binding of the one or more agents to a cell in the sample comprises flow cytometry.

38. The method of one of claims 33-37, further comprising treating the subject with a determined presence of tumor-infiltrating Treg cells in the tumor environment with a treatment to inhibit or deplete the tumor-infiltrating Treg cells.
